SEQUENCE LISTING (32 sequences)

SEQ ID NO: 1 - DNA, 140 bp, Anabaena sp.
ggggtattcgctatcagtcttggcgctactgcccatcccgcccctcaaacctttgtccgtccgcctaagactgataccgctactggtgacaggccgatgttatatctggagttctatgtctttttctcatctcggcttgt

SEQ ID NO: 2 - DNA, 238 bp, Anabaena sp.
ggggtattcgctatcagtcttggcgctactgcccatcccgcccctcaaacctttgtccgtccgcctaagactgataccgctactggtgacaggccgatgttatatctggagttctatgtctttttctcatctcggcttgtccaatgaaattatcaatgctgttactgagttggggtacaccaaacccacacccatccagatgcagtctattcctgctgtcttatcaggacgagatttg

SEQ ID NO: 3 - DNA, 14 bp, Artificial
  Primer for Polymerase Chain Reaction (PCR)
tgtcagttgctact

SEQ ID NO: 4 - DNA, 15 bp, Artificial
  Primer for PCR (Table 2)
ccatgagcgcattca

SEQ ID NO: 5 - DNA, 19 bp, Artificial
  Primer for PCR (Table 2)
cgtcctgataagacagcag

SEQ ID NO: 6 - DNA, 20 bp, Artificial
  Primer for PCR (Table 2)
taatacgactcactataggg

SEQ ID NO: 7 - DNA, 18 bp, Artificial
  Primer for PCR (Table 2)
gggtgtgggtttggtgta

SEQ ID NO: 8 - DNA, 21 bp, Artificial
  Primer for PCR (Table 2)
ggctggttgtagagatcttgg

SEQ ID NO: 9 - DNA, 23 bp, Artificial
  Primer for PCR (Table 2)
ggacaagccgagatctgaaaaag

SEQ ID NO: 10 - DNA, 22 bp, Artificial
  Primer for PCR (Table 2)
ggccagtgagcgcgcgtaatac

SEQ ID NO: 11 - DNA, 20 bp, Artificial
  Primer for PCR (Table 2)
ggggagagtaaagctggcag

SEQ ID NO: 12 - DNA, 21 bp, Artificial
  Primer for PCR (Table 2)
ctctgttaggatccaccattg

SEQ ID NO: 13 - DNA, 22 bp, Artificial
  Primer for PCR (Table 2)
gcgccagctagcaaatctcgtc

SEQ ID NO: 14 - DNA, 17 bp, Artificial
  Primer for PCR (Table 2)
ggacaagccgagatgag

SEQ ID NO: 15 - DNA, 16 bp, Artificial
  Primer for PCR (Table 2)
ggggtgcaatcttcgc

SEQ ID NO: 16 - DNA, 18 bp, Artificial
  Primer for PCR (Table 2)
cgtcatttccaactatta

SEQ ID NO: 17 - DNA, 16 bp, Artificial
  Primer for PCR (Table 2)
cctgtactctgttaag

SEQ ID NO: 18 - DNA, 19 bp, Artificial
  Primer for PCR (Table 2)
gatagcgaataccccaatg

SEQ ID NO: 19 - DNA, 18 bp, Artificial
  Primer for PCR (Table 2)
ggtttgaggggcgggatg

SEQ ID NO: 20 - DNA, 16 bp, Artificial
  Primer for PCR (Table 2)
cttaacagagtacagg

SEQ ID NO: 21 - DNA, 18 bp, Artificial
  Primer for PCR (Table 2)
ttccaacgattaagatta

SEQ ID NO: 22 - DNA, 22 bp, Artificial
  Primer for PCR (Table 2)
ccccaatggtagatcttaacag

SEQ ID NO: 23 - DNA, 22 bp, Artificial
  Primer for PCR (Table 2)
catgaattctttaggggctggt

SEQ ID NO: 24 - DNA, 21 bp, Artificial
  Primer for PCR (Table 2)
cccgaattctgaatcttaaca

SEQ ID NO: 25 - DNA, 55 bp, Artificial
  Primer for PCR (Table 2)
gcgaattcggatccaactattaatattaaagtttagagaaaggatccgaattcgg

SEQ ID NO: 26 - DNA, 55 bp, Artificial
  Primer for PCR (Table 2)
ccgaattcggatcctttctctaaactttaatattaatagttggatccgaattcgc

SEQ ID NO: 27 - DNA, 22 bp, Artificial
  Primer for PCR (Table 2)
gttttcccagatctagtttgag

SEQ ID NO: 28 - DNA, 22 bp, Artificial
  Primer for PCR (Table 2)
cgcctaagacggatcccgctac

SEQ ID NO: 29 - DNA, 18 bp, Artificial
  Primer for PCR (Table 2)
ggtttgaggggcgggatg

SEQ ID NO: 30 - DNA, 20 bp, Artificial
  Primer for PCR (Table 2)
gcttcccaaccttaccagag

SEQ ID NO: 31 - DNA, 22 bp, Artificial
  Primer for PCR (Table 2)
caccaaaattaatggattgcac

SEQ ID NO: 32 - DNA, 29 bp, Artificial
  Primer for PCR (Table 2)
cggtagagttgcccctactccggttttag

(19) United States
(12) Patent Application Publication
(10) Pub. No.: US 2009/0042301 A1
(43) Pub. Date: Feb. 12, 2009

(54) TEMPERATURE REGULATED GENE EXPRESSION

(75) Inventors: George OWTTRIM, Edmonton (CA); Danuta CHAMOT, Edmonton (CA); Jessica M. BROWN, Edmonton (CA)

Correspondence Address: EDWARD YOO C/O BENNETT JONES, 1000 ATCO CENTRE, 10035 - 105 STREET, EDMONTON, ALBERTA, AB, T5J3T2, CA

(73) Assignee: THE GOVERNORS OF THE UNIVERSITY OF ALBERTA, Edmonton (CA)

(21) Appl. No.: 11/675,722

(22) Filed: Feb. 16, 2007

Related U.S. Application Data
(60) Provisional application No. 60/766,889, filed on Feb. 16, 2006.

(51) Int. Cl. C12N 15/87; C12N 15/11; C12N 15/00; C12N 1/20

(52) U.S. Cl. 435/471; 435/440; 536/24.1; 435/320.1; 435/252.3; 435/252.33; 435/252.32

(57) ABSTRACT

A method for regulation of gene expression by variation of temperature uses a riboswitch. The riboswitch includes a 5′-UTR construct of crhC which alters its secondary structure in response to temperature, resulting in a more stable transcript at lower temperatures, permitting translation. At higher temperatures, the transcript is destabilized and functionally inactive. The 5′-UTR construct of crhC may be operatively linked to a promoter and a gene and administered to cells with an expression vector.

### CROSS REFERENCE TO RELATED APPLICATIONS

The present application claims priority benefit of U.S. Provisional Patent Application No. 60/766,889 filed on Feb. 16, 2006, the contents of which are incorporated herein by reference.

### FIELD OF THE INVENTION

The invention relates to the regulation of gene expression under conditions of physiologic stress, and in particular, the regulation of gene expression by temperature variation using a riboswitch associated with the cold shock response of a bacterium.

### BACKGROUND

The regulation of bacterial gene expression occurs at many levels, including transcriptional control, or control of the synthesis of mRNA from a given gene; translational control, or the regulation of the efficiency by which the mRNA is translated into polypeptide sequence by the ribosome; and mRNA stability, or the efficiency at which a given mRNA population within the cell is degraded and rendered inactive.

Microorganisms encounter various levels of stress every day including osmotic, alkaline, acid, nutrient, and thermal stress. The organism's survival depends on the ability to adapt rapidly and specifically to external stimuli, permitting growth under stressful conditions. One of the major stresses encountered by microorganisms is a downshift in temperature, below the optimal growth temperature. This is referred to as cold shock or cold stress and is characterized by a limited number of specific changes in cell physiology as a result of alterations in gene expression in response to reduced temperature. Cold stress is species-specific; what is cold to one species may be optimal for another. For example, a drop in temperature greater than 13° C. from E. coli 's optimal growth temperature (37° C.) is considered cold shock whereas in Anabaena, a drop of greater than 5° C. from its optimal laboratory growth temperature (30° C.) is sufficient to elicit the cold shock response.

During the cold shock response, each cell goes through two growth phases, an initial lag phase called the acclimation phase, and the recovery phase where normal growth resumes after adaptation. During the acclimation phase, many physiological changes occur within the cell, including a decrease in membrane lipid saturation, an increase in mRNA stability, hindered and/or altered ribosomal function, misfolding of proteins, increase in DNA superhelicity, and inhibition of DNA, RNA and protein synthesis. In response to the cold-induced cellular and physiological constraints, the cell reprograms gene expression, activating specific cold shock (CS) genes that encode cold shock proteins (CSPs). Thus, the cold shock response governs the expression of CSPs whose function allows adaptation to low temperatures.

CSPs are a diverse group of proteins involved in cellular cold acclimation processes involving transcription, translation, and mRNA function. Most CSPs bind nucleic acids and have a diverse range of functions including alteration of nucleic acid secondary structure, antitermination of transcription, ribosome assembly and association, and mRNA degradation. Some prototypical CSPs include lipid desaturases, RNA chaperones, RNA helicases, proteases, and proteins involved in the transcriptional and translational machinery. The most extensively studied CSP is the major cold shock protein in E. coli, CspA. CspA comprises a large gene family (CspA-I) of small (˜7.5 kD) proteins, four of which are cold-inducible (CspA, CspB, CspG, and CspI). The Csp protein family has a high degree of homology with CSPs from several other species including, Bacillus subtilis, Bacillus cereus, and the eukaryotic Y-box proteins, but are absent from cyanobacteria. The identification of homologous CSPs throughout various prokaryotic and eukaryotic systems leads to the assumption that the Csp protein family members play a crucial role in acclimating to reduced growth temperature.

Two different classes of CSPs have been identified: Class I, whose expression is dramatically increased upon a temperature downshift, and Class II, whose expression is constitutively low at optimal growth temperatures with a marginal increase in expression in the cold. Class I CSPs include proteins such as, DesB and DesD from Synechocystis, and CrhC from Anabaena. Class II CSPs include DesC and CrhR from Synechocystis and CrhB from Anabaena (Chamot et al., 1999). Thus, the levels of cold shock gene expression vary depending on gene regulation and the protein's cellular role in adapting to low temperatures.

In contrast to E. coli and other bacteria in which the Csp family of CSPs constitutes the major cold shock protein, cyanobacteria do not possess homologues of the csp genes. It has been proposed that a family of RNA chaperones encoding RNA-binding proteins (Rbps) replaces the Csp family in cyanobacteria. RNA-binding proteins bind to RNA with high affinity for polyU and polyG sequences and are involved in various aspects of RNA metabolism such as modification, maintenance of stability, and translation. Although the cold shock domain (CSD) and the ribosome-binding (RBD) domain of the Rbps differ in topology and sequence from Csps, research suggests that they may have a converged function with respect to the cold shock response and that the Rbps may be the cyanobacterial counterpart of E. coli CSPs.

Two RNA helicases, CrhB and CrhC, have also been identified in Anabaena sp. strain PCC 7120 (Chamot et al., 1999). CrhC is a CSP as it was shown to be strictly cold-inducible upon a temperature shift from 30° C. to 20° C., whereas CrhB is expressed under a broad range of conditions, with enhanced expression in the cold (Chamot et al., 1999).

RNA helicases can be involved in any cellular process that involves modulation of RNA secondary structure. RNA helicases are encoded in almost all species (except for a few bacteroids) and aid in essential cellular functions by unwinding double-stranded or duplexed RNA into a single-stranded, functional form. Using PCR cloning and degenerate primers modeled after conserved sequences in the tobacco eIF-4A gene family, Chamot et al. (1999) identified two putative cyanobacterial RNA helicases (Crh), CrhB and CrhC, which are differentially expressed in Anabaena. CrhB and CrhC were the first RNA helicases to be identified and characterized in cyanobacteria. Based on spatial and sequence similarities to eIF-4A, both cyanobacterial RNA helicases are believed to belong to the DEAD-box family with CrhB showing 51% similarity to CrhC. CrhC showed sequence similarity to a large majority of known RNA helicases, particularly to the E. coli RNA helicase RhlE, with 74% similarity (Chamot et al., 1999).

CrhC is a 47 kDa protein containing seven of the eight conserved helicase motifs with a novel FAT box (Phe-Ala-Thr) replacing the conserved SAT motif (Ser-Ala-Thr) (Chamot et al., 1999). The SAT motif is required for helicase unwinding activity therefore an amino acid modification (S to F) may imply changes to CrhC activity and substrate specificity. The modified SAT to FAT motif introduces a hydrophobic, aromatic phenylalanine residue, which is not capable of normal serine hydrogen bond formation to produce a more rigid protein. This unique property of CrhC may specify RNA substrate interactions, specify accessory protein interactions, or may allow for CrhC to unwind RNA in a unidirectional fashion (Chamot et al., 1999; Yu and Owttrim, 2000).

CrhC expression is solely temperature-regulated, with transcript accumulation occurring only during cold stress and not in response to a range of other abiotic factors (Chamot et al., 1999). The crhC transcript is not detectable at 30° C., but following a downshift in temperature to less than 25° C. (cold stress) there is a rapid accumulation of crhC transcript. crhC transcript levels remain elevated during cold stress but return to basal levels upon a return to 30° C. (Chamot and Owttrim, 2000; Chamot et al., 1999). The CrhC protein expression profile follows a similar pattern as the crhC transcript profile, with CrhC present only in cold-shocked cells.

Due to the expression patterns of CrhB and CrhC, both RNA helicases are believed to play a unique and specific role in cold acclimation. Proposed mechanisms include unwinding RNA secondary structure in the 5′ UTR of cold-induced mRNAs thereby removing the block in translation initiation. CrhC-induced RNA unwinding activity could therefore initiate the translation of specific CSP mRNAs, whose protein products alleviate the physiological constraints imposed by a temperature downshift.

Temperature-dependent expression of CrhC may be regulated by conserved elements found within the translation initiation region. A putative downstream box (DB) (+137 to +151) was identified within the crhC open reading frame (ORF) having sequence complementation to the Anabaena 16S rRNA. The E. coli DB is predicted to be involved in the formation of extended complementary base pairing around the Shine-Dalgarno (SD) sequence thereby enhancing translation initiation.

crhC has a long (115 nt) 5′ UTR that is predicted to fold into a stable secondary structure. The translation start site (AUG) is found in a loop whereas the SD sequence and the DB are proposed to form a partial, base pairing stem (Chamot et al., 1999). Finally, downstream of the translation stop codon is a putative 22 bp stem loop followed by a stretch of U residues, a structure characteristic of a Rho-independent transcriptional terminator. This 3′UTR structure could provide crhC with not only a mechanism for terminating transcription, but may also be involved in mRNA stability.

Comparably, the crhC sequence harbors conserved cis-acting elements identified in cspA as contributing to temperature-regulated expression. The reoccurrence of cold-regulating cis-acting elements throughout different species may suggest similar or even global regulatory mechanisms involved in regulating cold shock gene expression.

Research suggests that CrhC interacts in vivo with accessory proteins either to provide substrate specificity or to optimize functionality during cold stress.

CrhC is differentially expressed in responses to cold stress, therefore coordinated regulation is necessary for activation and inactivation of CrhC expression at low and optimal growth temperatures, respectively. CrhC expression is strictly dependent on a temperature downshift, with transcript and protein accumulation occurring constitutively at 20° C. (cold stress) but not at 30° C. (optimal growth) (Chamot and Owttrim, 2000; Chamot et al., 1999). Chamot and Owttrim (2000) established that crhC transcript accumulation was temperature-dependent, with regulation that may occur at the transcriptional, post-transcriptional, mRNA stability, and translational levels.

### SUMMARY OF THE INVENTION

It has been discovered that the 5′-UTR of crhC mRNA alters its secondary structure in response to temperature changes, resulting in a more stable transcript at lower temperatures, permitting translation of crhC. At higher temperatures, the transcript is destabilized, possibly because the 5′-UTR of crhC is recognized by an RNase, or possesses intrinsic RNA cleavage activity i.e. ribozyme activity. In particular, it is believed that the secondary structure of a 5′ stem-loop structure, but not a 3′ stem-loop structure, in the 5′ UTR of crhC changes under cold shock conditions in Anabaena. As a result, the 5′ UTR of crhC may be used to regulate expression of a gene by temperature variation.

Therefore, in one aspect, the present invention comprises a nucleic acid molecule that regulates expression of a gene under conditions that elicit the cold shock response in Anabaena, said nucleic acid molecule comprising a 5′-UTR construct of crhC operably linked to a non-native promoter.

In one general aspect, the invention provides a method of regulating expression of a gene by inducing temperature changes, comprising the steps of:
a) administering to the cell a heterologous nucleic acid molecule, comprising a promoter operably linked to a 5′-UTR construct of crhC and a coding sequence of the gene; and
b) controlling the temperature environment of the cell.

The 5′ UTR construct encodes an RNA, referred to herein as a riboswitch, which comprises a nucleotide sequence having a first secondary structure at one temperature, and a second secondary structure at a lower temperature, wherein the lower temperature elicits the cold shock response in Anabaena, and wherein the first secondary structure is functionally inactive, while the second secondary structure is functionally active, leading to ribosomal loading and translation of the associated open reading frame.

In another aspect, the invention may comprise a nucleic acid molecule for regulation of gene expression comprising a promoter operatively linked to a 5′-UTR construct of crhC and a coding sequence of the gene. The 5′-UTR construct encodes an RNA having a first secondary structure at a first temperature and a second secondary structure at a second temperature; wherein the first secondary structure is associated with suppression of gene expression while the second secondary structure is associated with active expression of the gene, and wherein the second temperature elicits the cold shock response in Anabaena.

In another aspect, the invention may comprise an expression vector comprising an isolated nucleic acid described herein, or a cell comprising such an expression vector. The cell may preferably be a bacterium, such as E. coli.

### BRIEF DESCRIPTION OF THE DRAWINGS

FIG. 1: Proposed models illustrating temperature-induced stabilizing/destabilizing of the crhC 5′ UTR, in relationship to the MFOLD results.

FIG. 2: (A) Plasmid pWM75-2, containing a 939 bp EcoR V fragment coding for the promoter, 5′ UTR, and 510 bp of the crhC ORF, used as the source of the JW series of deletions. (B) Plasmid pWM75-3 containing a 2424 bp Hinc II insert containing the full-length crhC gene (promoter, 5′-UTR, ORF, and the 257 bp 3′-UTR) cloned into pBluescript KS+.

FIG. 3: (A) Construction of the plasmid pJBm1 from the pSIG16 vector backbone. (B) Cloning of the 5′-UTR, ORF and 3′-UTR of crhC into BamH I of pSIG16 to create pJBm1, which therefore lacks the natural promoter of crhC.

FIG. 4. Western and Northern blot analysis of pJBm1 in E. coli demonstrating cold-induced temperature-dependent expression of crhC at both the transcript and protein level.

FIG. 5. MFOLD prediction of the structure of a 141 nt RNA fragment, containing the entire crhC 5′ UTR extending 26 nt into the ORF, at 30° C.

FIG. 6. MFOLD prediction of the structure of a 141 nt RNA fragment, containing the entire crhC 5′ UTR extending 26 nt into the ORF, at 24° C.

FIG. 7. Diagrammatic representation of the MFOLD prediction of the crhC 5′ UTR secondary structure at 30° C. (A) and 24° C. (B).

FIG. 8. Cloning of various combinations of the 5′ UTR stem-loop structures between a medium strength E. coli constitutive promoter and the lux operon. (A) pSIG11 lux plasmid backbone. (B) Construction of plasmid pJBm3 containing both of the 5′ UTR stem-loop structures. (C) Construction of plasmid pJBm4 by cloning only the 3′ UTR stem-loop structure into pSIG11.

FIG. 9. Both the 3′ and 5′ stem-loop structures of the crhC 5′ UTR are required to convey temperature-dependent expression of lux in E. coli; luciferase activity observed for pSIG11, pJBm3, and pJBm4, following transfer to reduced temperature (20° C.).

FIG. 10. The crhC promoter alone does not convey temperature-dependent expression to lux in E. coli.

FIG. 11. The crhC promoter combined with the 5′ UTR extending 11 nt into the ORF does not convey temperature-dependent expression to lux in E. coli.

FIG. 12. The full-length crhC gene, transcriptionally fused to the lux operon, conveys temperature-dependent expression to lux in E. coli.

FIG. 13. Recognition of a distinct cleavage product of the crhC 5′ UTR with natural self-cleavage in ribozyme reactions performed at 37° C. and 30° C. but not at 20° C. (i.e. cold stress).

### DETAILED DESCRIPTION OF PREFERRED EMBODIMENTS

When describing the present invention, all terms not defined herein have their common art-recognized meanings. To the extent that the following description is of a specific embodiment or a particular use of the invention, it is intended to be illustrative only, and not limiting of the claimed invention. The following description is intended to cover all alternatives, modifications and equivalents that are included in the spirit and scope of the invention, as defined in the appended claims.

Bacterial growth at reduced temperatures results in major cellular constraints that are adjusted by the expression of a specific set of genes, the cold shock (CS) genes. The cold shock response in the cyanobacterium Anabaena is induced following a temperature downshift of greater than about 5° C., from its optimal growth temperature (30° C.) (Chamot et al., 1999; Yu and Owttrim, 2000). The present invention involves a CS gene believed to be involved in removing the block in translation initiation. CrhC, a cold-induced RNA helicase from Anabaena, was proposed to alleviate inhibitions in translation initiation by unwinding stable RNA secondary structures formed at low temperature (Chamot and Owttrim, 2000; Yu and Owttrim, 2000).

Recent studies on CS gene regulation indicate that bacteria use a variety of post-transcriptional regulatory mechanisms to coordinate gene expression. It has been demonstrated with half-life studies that the crhC transcript was stabilized (6×) at reduced temperatures (20° C.), providing evidence that mRNA stability is important for temperature-regulated crhC expression. We have demonstrated that a small amount of transcript and CrhC protein accumulate at 30° C. in E. coli. The presence of CrhC at 30° C. indicates that translation can occur at 30° C. and therefore, the limiting factor is the availability of functional transcript.

Investigation into the crhC 5′ UTR demonstrated that the 5′ UTR is involved in post-transcriptionally regulating crhC expression. Transcriptional reporter fusions constructs demonstrated that the 5′ UTR was necessary and sufficient to convey temperature-dependent expression as long as both stem loop secondary structures were present. Using MFOLD predictions combined with in vivo expression patterns in E. coli, it was hypothesized that temperature-induced structural changes within the crhC 5′ UTR stabilized the transcript at 20° C. whereas the 5′ UTR structure at 30° C. destabilized the transcript via intrinsic ribozyme activity or endogenous RNase activity. Temperature-induced structural alterations of the mRNA suggest that the crhC 5′ UTR likely acts as a thermosensor, regulating mRNA stability or destability.

Accordingly, without restriction to a theory, the 5′ UTR of crhC can be used as a thermoresponsive regulator of gene expression, referred to herein as a riboswitch. When paired with a promoter and a heterologous gene in an expression system, the gene may be turned on or off by temperature variations. As used herein, a 5′ UTR construct comprises a nucleotide sequence which encodes a functional riboswitch RNA sequence beginning at the 5′ transcription start site (i.e. the 5′ end of the mRNA) and extending downstream to the ATG translation start codon (underlined below). In one embodiment, the 5′ UTR construct comprises a sequence beginning at the 5′ transcription start site and extending 22 nucleotides downstream of the ATG translation start codon, as shown below in SEQ ID NO: 1. In another embodiment, the sequence extends up to a convenient NheI restriction endonuclease site, as shown below in SEQ ID NO: 2:

Construct 1: 22 nucleotides downstream of the ATG translation start codon [SEQ ID NO: 1]
ggggtattcgctatcagtcttggcgctactgcccatcccgcccctcaaacctttgtccgtccgcctaagact
gataccgctactggtgacaggccgatgttatatctggagttctatgtctttttctcatctcggcttgt

Construct 2: up to the NheI restriction endonuclease site [SEQ ID NO: 2]
ggggtattcgctatcagtcttggcgctactgcccatcccgcccctcaaacctttgtccgtccgcctaagact
gataccgctactggtgacaggccgatgttatatctggagttctatgtctttttctcatctcggcttgtccaa
tgaaattatcaatgctgttactgagttggggtacaccaaacccacacccatccagatgcagtctattcctgc
tgtcttatcaggacgagatttg

The riboswitch function of a 5′ UTR construct resides in its secondary structure and not its actual nucleotide sequence. Therefore, variations in the sequence which do not substantially affect the secondary structure and its temperature-induced alteration are within the scope of the present invention. Accordingly, in one embodiment of the present invention, a 5′UTR construct composition comprises a nucleic acid sequence which encodes a functional riboswitch and having at least about 60% homology with the nucleotide sequence of SEQ ID NO: 1 or SEQ ID NO: 2. In another embodiment of the present invention, the isolated nucleic acid composition comprises a nucleic acid sequence which encodes a functional riboswitch and having at least about 75% homology with the nucleotide sequence of SEQ ID NO: 1 or SEQ ID NO: 2. Preferably, the degree of homology is greater than 90%. In still another embodiment of the present invention, the isolated nucleic acid composition comprises a nucleic acid sequence that is substantially the same as, or identical to, the nucleotide sequence of SEQ ID NO: 1 or SEQ ID NO: 2.

The role of the 5′ UTR in temperature-dependent crhC expression was demonstrated in E. coli, where the crhC gene (lacking its own promoter) was placed under the transcriptional control of a constitutive E. coli promoter (pSIG16). The observation that transcript and protein accumulation was cold-induced and not constitutive indicated that post-transcriptional events are regulating crhC expression. These results confirm that transcriptional regulation was not required for temperature-dependent expression rather, crhC is differentially regulated by transcript stabilization at reduced temperatures.

RNA secondary structure may be accurately predicted by computer models. Dynamic programming methods of prediction are well known and include MFOLD (http://bioweb.pasteur.fr/seqanal/interfaces/mfold-simple.html; Zuker and Stiegler, 1981; Zuker et al., 1999). Based on the minimal free energy (AG), MFOLD predicted that the crhC 5′ UTR contained two cis-acting stem loops that exist in different secondary structures at 30° C. and 24° C. MFOLD predicted that at temperatures ≧25° C. the 5′ UTR would fold in one RNA secondary formation whereas at temperatures ≦24° C. an altered RNA secondary structure would form. Surprisingly, a difference of 1° C. (25° C. to 24° C.) as predicted by MFOLD, was capable of thermodynamically altering the crhC 5′ UTR. It has been previously demonstrated (Chamot and Owttrim (2000)) that the threshold temperature for crhC transcript accumulation was in the vicinity of 25° C., which correlates with the MFOLD predicted temperature where altered RNA secondary structure occurs. Following a downshift in temperature, extensive secondary structure was predicted within the 5′ stem loop structure of the crhC 5′ UTR whereas no temperature-induced alterations were observed in the 3′ stem loop structure.

From these results, without being bound by a theory, it is believed that low temperature-induced alterations in the crhC 5′ UTR stabilize the transcript, permitting ribosomal loading and translation of crhC at 20° C. In relationship to the crhC translational regulatory elements, the SD sequence and DB (no UB consensus sequence was found) are all located within the 3′ stem loop structure suggesting that structural inhibition of ribosome access for translation initiation is not a regulatory factor. Indeed, no temperature-induced structural changes in the 3′ stem loop were predicted by MFOLD. Therefore, the 3′ stem loop structure alone is not sufficient to differentiate between 20° C. and 30° C., as no thermodynamic structural changes were predicted to allow for differential ribosome access to the SD and DB. Based on the above observations, CrhC's differential regulation does not appear to originate from temperature-induced alterations in ribosomal loading capacities. The 5′ UTR sequence is both necessary and sufficient to convey temperature-regulated expression of heterologous genes in heterologous systems. The crhc mRNA (or transcript) is physically not present at temperatures above 25° C., not simply inactive.

Cold-induced stabilization of the crhC transcript is triggered by local changes within the secondary structure of the 5′ loop, reducing its targeted degradation. In contrast, it is believed that the 5′ UTR secondary structure at temperatures above 24° C. leads to the inactivation of the crhC mRNA either through interactions with cellular factors (i.e. RNases) which either cleave or bind the RNA, or by self-cleavage via intrinsic ribozyme activity. The present invention is not limited to either theory, as the mechanism of inactivation is not a limiting factor. Both theories of inactivation are within the scope of the present invention.

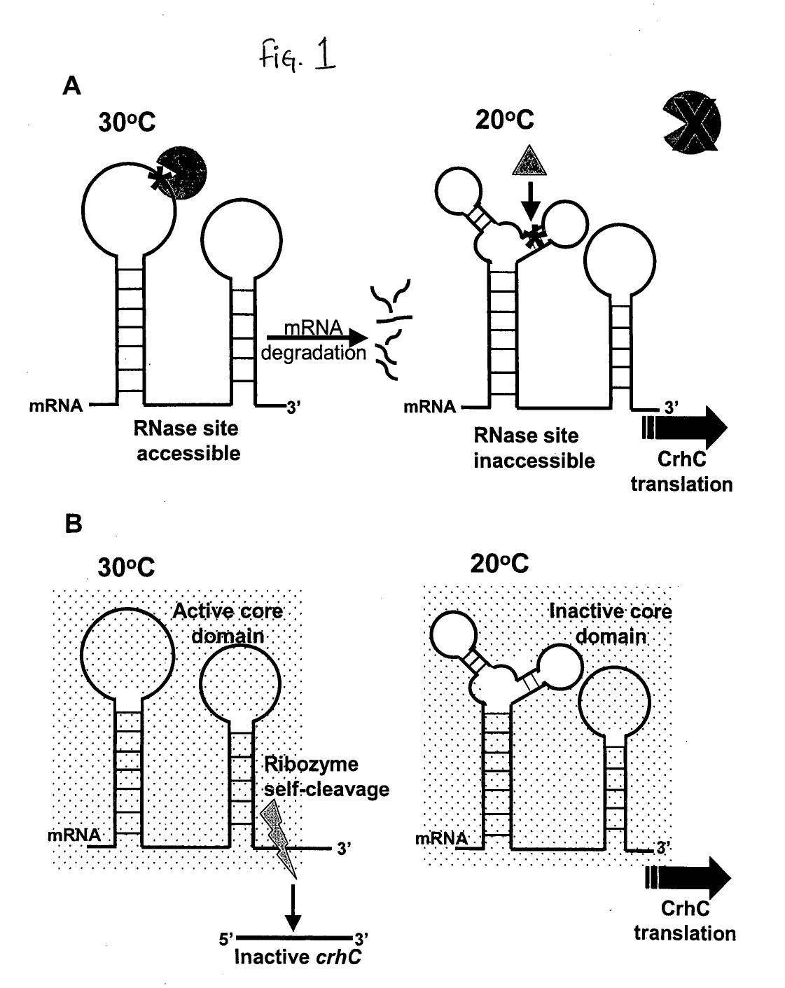

Proposed models illustrating temperature-induced stabilizing/destabilizing of the crhC 5′ UTR, in relationship to the MFOLD results, are shown in FIG. 1. FIG. 1A illustrates that at optimal growth temperatures (30° C.), base pairing of the crhC 5′ loop is “melted” creating a 18 nt unpaired region potentially accessible for degradation by ribonucleases (RNases), first by endonucleases such as RNase E, followed by exonuclease digestion (e.g. RNase R, PNPase, RNase II) (Cairrao et al., 2003). Constriction of the crhC 5′ loop at reduced temperature (≦24° C.) may stabilize the transcript by masking endoribonuclease target site(s) and by inhibiting RNA degradation. The inability of specific ribonucleases or the RNA degradosome to initiate crhC mRNA degradation due to increased 5′ UTR secondary structure or the inactivation of some mRNA degradation machinery at low temperature (Goldenberg et al., 1996), or both, may also contribute to providing mRNA stability to crhC during cold stress. mRNA stability of CS genes can also be altered by interacting with specific nucleic acid binding proteins (FIG. 1A). Potentially, the thermodynamic alterations predicted within the crhC 5′ UTR could provide access to, or mask, a recognition site(s) for RNA binding proteins, stabilizing or destabilizing the transcript. For example, during cold stress, the altered (constricted) secondary structure of the 5′ loop could create a protein recognition site, allowing an RNA binding protein to bind and stabilize the transcript, and/or potentially facilitate translation initiation. In this context, it is interesting to note that cyanobacteria encode a family of RNA binding proteins, the Rbp gene family, whose expression is temperature-regulated (Sato et al., 1995). These proteins have been proposed to functionally replace the csp gene family, which have been proposed to function as RNA chaperones and it is possible that they may be similarly involved in stabilization of cold shock mRNAs in cyanobacteria.

Understanding crhC transcript destabilization at 30° C. appears to be complex. The ability of the crhC promoter to activate temperature-induced transcription at 30° C. suggests that crhC transcription is constitutive and independent of temperature. Thus, the absence of crhC transcript at 30° C. indicates that the crhC transcript is being actively degraded. Inhibition of translation by RNA secondary structure also does not appear to play a role here, as limited protein accumulation is observed at 30° C. when crhC was cloned on a high copy plasmid, thus implying that crhC mRNA is translationally active at all temperatures. crhC mRNA must therefore be actively degraded at temperatures above 24° C. The mechanism(s) destabilizing crhC presumably involves recognition of the temperature-induced secondary structure formed at 30° C. and is initiated by primary cleavage by two possible mechanisms (FIG. 1): In one case, an endogenous RNase recognizes the secondary structure at 30° C. but not 20° C. AND is conserved between cyanobacteria and E. coli, which is the heterologous system in one embodiment of the invention. In another possibility, the RNA secondary structure of the 5′ UTR which spontaneously forms at 30° C. possesses intrinsic RNA cleavage activity (i.e. ribozyme activity).

Evidence suggests that the crhC 5′ UTR may possess intrinsic ribozyme activity, activated by temperature cues. A significant difference was noted between the crhC 5′ UTR and control RNA degradation patterns. Results show that at 30° C. the crhC 5′ UTR degradation appeared somewhat non-random, indicating intrinsic ribozyme activity.

In support of crhC destabilization via ribozyme activity, preliminary analysis of temperature-regulated CrhC protein accumulation in E. coli RNase mutants, including Δrnc, Δpnp, and Δrne, does not identify altered CrhC expression. If one of these RNases was required for the initial cleavage, constitutive CrhC expression would be detected at temperatures above 25° C. These results suggest that these RNases are not involved in the initial cleavage of the crhC transcript at 30° C.

In summary, in view of these results, it is believed that the crhC 5′ UTR may function as a ribozyme whose secondary structure produces an active RNA enzyme at 30° C. and not at 20° C. crhC 5′ UTR function as a ribozyme at an elevated temperature is unique as it does not involve an effector molecule to alter secondary structure. Rather, temperature-induced structural changes within the 5′ UTR appears to regulate ribozyme activity.

The presence of temperature responsive cis-elements within the crhC 5′ UTR suggests that the 5′ untranslated region of crhC may act as a “cellular thermometer”, which may be referred to as a “riboswitch”. Thermosensing mechanisms have been identified at both the transcriptional and translation level and can involve several different events; changes in DNA supercoiling, membrane fluidity, mRNA confirmation, and protein confirmation. It has been discovered that crhC's thermoregulation occurs primarily due to thermosensitive structural alteration of RNA secondary structure within the 5′ UTR. Such a mechanism allows essentially instantaneous response to temperature change with no requirement for indirect temperature sensing, signal transduction, activation of transcription and finally translation. RNA transcript is either unstable or stable, and when stable, it is translationally competent.

### Examples

The following examples are provided to illustrate aspects of the invention and are not to be construed in any way as limiting the scope of the invention, which is defined in the appended claims.

1.1. Bacterial Strains and Growth Conditions

The bacteria identified herein, their relevant genotypes and their sources are listed in Table 1. Herein, bacteria harboring plasmids will be designated with the bacterial strain first followed by the plasmid (p) in parentheses [for example, DH5α (PSIG11) denotes the strain DH5α containing plasmid pSIG11]. E. coli DH5α cells were grown in liquid LB media (Luria broth media containing 10 g/L bacto tryptone, 5 g/L yeast extract, 5 g/L NaCl, and buffered with 1 mL of 1N NaOH) and maintained on solid media containing 1.2% (w/v) bacto agar. When required, LB medium was supplemented with the appropriate antibiotics at the following concentrations: ampicillin 100 μg/mL, kanamycin 50 μg/mL. All E. coli strains were grown at 37° C., with liquid cultures aerated by shaking at 200×rpm. When cold shock treatment was required, liquid cultures were transferred to either 20° C. incubators (Coldstream) or a 20° C. water bath shaker for the indicated times, with shaking at 200×rpm.

Anabaena sp. strain PCC 7120 was maintained on agar plates composed of BG-11 (Allen, 1968), containing 1% (w/v) Difco grade Bacto-agar, grown in Coldstream incubators at 30° C. under constant illumination (Phillips Alto cool, white fluorescent light, 30 μmoles photons/m²/sec). Liquid cultures were aerated by bubbling with air and shaking at 200×rpm. When cold shock treatment was required, liquid cultures were transferred to a 20° C. Coldstream incubator, with bubbling and shaking, for the indicated times.

TABLE 1
Bacterial Strains, their relevant genotypes and their sources

| Strain | Relevant | Reference/Source | Use |
|---|---|---|---|
| Anabaena variabilis | Wild Type | University of Toronto | Study Subject |
| UTCC 387 | | Culture Collection | |
| (equivalent to | | (UTCC) | |
| Anabaena sp. strain | | | |
| PCC^{a} 7120) | | | |
| Synechocystis sp. strain | Wild Type | University of Toronto | Control |
| PCC^{a} 6803 | | Culture Collection | |
| | | (UTCC) | |
| Synechococcus sp. | Wild Type | University of Toronto | Control |
| strain | | Culture Collection | |
| PCC^{a} 7942 | | (UTCC) | |
| Escherichia coli DH5a | A (lacZYA-argF)_{U169} | Ausubel et al., 1995 | Plasmid propagation |
| | (m80 lacZ AM 15) | | |
| | hsdR17(r_{K}⁻m_{K}⁺) recAl | | |
| | F′/endAl | | |
| | thi-1 relAl gyrA | | |
| | supE44 | | |

^{a}Pasteur Culture Collection

1.2 Isolation and Purification of Plasmid DNA

1.2.1 Small Scale Plasmid Purification from E. coli

To isolate and purify small amounts of high copy plasmid DNA from E. coli, the TENS mini-prep method (Zhou et al., 1990) was employed. A 1.5 mL aliquot of a saturated overnight culture was harvested by microcentrifuging for 10 seconds at 14,000×g. The supernatant was decanted and the pellet resuspended in the remaining ˜100 μL media. The cells were lysed by vortexing for 1-2 seconds in 300 μL of TENS solution (100 mM Tris, pH 8.0, 1 mM EDTA, pH 8.0, 1 NaOH: 0.5% [w/v] SDS). Following lysis, the solution was neutralized by vortexing for 1-2 sec in 150 μL of 3 M sodium acetate, pH 5.1. Cellular debris was pelleted by microcentrifugation for 5 minutes and the supernatant transferred to a sterile microfuge tube. The plasmid DNA was precipitated by the addition of 900 μL of ice-cold 100% ethanol and immediately pelleted by microcentrifugation at room temperature for 5 minutes at 14,000×g. The pellet was washed with 1 mL of ice-cold 70% [v/v]ethanol and microcentrifuged for 5 minutes at room temperature. The pellet was air dried and resuspended in 50-100 μL sterile MilliQ (mQ) dH₂O.

To isolate and purify low copy number plasmids from E. coli using the TENS mini-prep protocol, a few alterations were made. A 6 mL aliquot of saturated overnight culture was harvested and the cells were lysed with 450 μL of TENS and 225 μL of 3M NaOAc, pH 5.1. Upon ethanol precipitation, the DNA pellets were resuspended in 30 μL of sterile mQdH₂O.

1.2.2 Large Scale Plasmid Purification from E. coli

High copy number plasmid DNA was isolated and purified from a 100 mL E. coli overnight culture using the QIAGEN plasmid Midi kit according to the manufacturer's protocol. Low copy number plasmid DNA was purified using the QIAGEN manufacturer's protocol suggested for purifying low copy number plasmids, using a 500 mL E. coli overnight culture.

1.3 Manipulation of DNA

1.3.1 Quantifying DNA

DNA was quantified by measuring the absorbance of a diluted sample at a wavelength of 260 nm. A 1 μL aliquot of the DNA sample was diluted in 500 μL of water, and the absorbance of the solution measured at 260 nm. The DNA concentration was determined using the extinction coefficient one absorbance unit is equivalent to 50 μg/mL of double-stranded (ds) DNA (1.0 A₂₆₀ nm=50 μg/mL dsDNA).

1.3.2 Digestion, Gel Electrophoresis, and Visualization of DNA

DNA was digested with restriction enzymes (RE) from New England Biolabs (NEB), Roche (Boehringer Mannheim), Invitrogen, Amersham, and Promega. In a 20 μL reaction, up to 5 μg of DNA was digested in 1× RE Buffer, as suggested by the manufacturer.

DNA fragments were separated and visualized on 0.7-1.4% [w/v] agarose (electrophoresis grade, ICN Biomedicals, Inc.) gels using either 0.5×TBE (45 mM Tris-borate, 1 mM EDTA) or 1×TAE (40 mM Tris-acetate, 1 mM EDTA) as the buffering system. One-fifth volume of 5×DNA loading buffer (30% [w/v] sucrose, 0.125% [w/v] bromophenol blue, 5 mM EDTA, pH 8.0) was added to each DNA sample prior to loading. Small-scale plasmid preparations were also treated with 1 μL of RNase A (10 mg/mL) at 37° C. for 10 minutes, to remove RNA contaminates. DNA agarose gels were electrophoresed at a constant voltage for the appropriate lengths of time, stained with ethidium bromide (10 μg/mL), and visualized and recorded digitally by observing fluorescence on a UV transilluminator (Syngene Genius Bio Imaging Systems). DNA fragment sizes were estimated by comparing the sample migration distances to known DNA standards (1 kb+ Ladder, Invitrogen), run simultaneously on each gel.

1.3.3 Purification of DNA from Agarose Gels

DNA fragments were purified from 1×TAE, 1% agarose gels using the PEG-trough method (Zhen and Swank, 1993). Upon electrophoretic separation and ethidium staining, a large majority of the 1×TAE (40 mM Tris-acetate, 1 mM EDTA) running buffer was removed and a UV hand-held illuminator (UVP Mineralight Lamp UVGL-58) was used to visualize the DNA. Below the DNA fragment of interest, a small cubic portion of the gel was excised with a clean scalpel, creating a trough. The trough was filled with PEG/TAE (18.75 mM PEG, 1×TAE) and a small amount of 1×TAE running buffer was returned to the electrophoresis apparatus, just enough to cover the bottom of the gel. With constant voltage, the DNA fragment of interest was electrophoresed into the PEG/TAE trough. The PEG/TAE containing the “trapped” DNA fragment was removed from the trough and extracted once with an equal volume of phenol:chloroform (1:1) and once with an equal volume of chloroform:isoamyl alcohol (24:1). The DNA was precipitated with 1.5× ice-cold 100% ethanol and washed with 1 mL of 70% ethanol. The air-dried pellet was resuspended in mQdH₂O, quantified (Example 1.3.1), and stored at −20° C.

1.3.4 Polymerase Chain Reaction (PCR)

DNA fragments were amplified using PCR, with reactions comprised of a combination (written as forward primer: reverse primer) of primer pairs (Table 2) and the appropriate template DNA. DNA fragments were amplified using the Expand Long Template PCR System (Roche) in either 50 or 100 μL final volumes. Each 50 μL reaction was comprised of 1×PCR Buffer #1 (1.75 mM MgCl₂), 350 μM of each dNTP (A, T, G, C), 10-20 pmoles of the required forward and reverse primer, 4 units of Expand DNA polymerase enzyme, and up to 20 fmoles of template DNA. After thorough mixing, the reaction was overlaid with an equal volume of mineral oil and amplified in a MiniCycler (MJ Research) using the specified Touchdown program. After the initial denaturation (94° C.), the annealing temperature decreases by 10° C., in 0.5° C. increments, over 20 cycles. The program cycles 20 more times at the lowest annealing temperature before completing amplification. The annealing temperature of each PCR reaction was determined based on the Tₘ of the primers as determined by the supplier (Sigma Genosys). A “hot start” was performed for all PCR reactions in which, the thermocycler was allowed to reach the initial denaturation temperature (94° C.) before inserting the reaction tubes.

TABLE 2

Oligonucleotide Primers

| | | Origin of | |
|---|---|---|---|
| Primer | Sequence (5′-3′) | Sequence | Use |
| WCM1 | TGTCAGTTGCTACT | crhC bp | EMSA non- |
| | [SEQ ID NO: 3] | 1031 to | competitive |
| | | 1018 | assays |
| | | (antisense | |
| | | strand) | |
| GWO36 | CCATGAGCGCATTCA | crhC bp 920 | EMSA non- |
| | [SEQ ID NO: 4] | to 933 | competitive |
| | | (sense | assays |
| | | strand) | |
| GWO43 | CGTCCTGATAAGACAGCAG | crhC bp 231 | Promoter cutback |
| | [SEQ ID NO: 5] | to 213 | identification/ |
| | | (antisense | Sequencing |
| | | strand) | |
| GWO71 | TAATACGACTCACTATAGGG | pBluescript | Biotinylated T7 |
| | [SEQ ID NO: 6] | KS+ | for DNA affinity |
| | | | chromatography |
| GWO72 | GGGTGTGGGTTTGGTGTA | crhC bp 193 | End- labeling |
| | [SEQ ID NO: 7] | to 176 | |
| JB1 | GGCTGGTTGTAGAGATCTTGG | crhC bp −391 | crhC promoter |
| | [SEQ ID NO: 8] | to −371 | transcriptional |
| | | (sense | fusion to the lux |
| | | strand) | operon |
| JB2 | GGACAAGCCGAGATCTGAAAAAG | crhC bp 142 | crhC promoter + |
| | [SEQ ID NO: 9] | to 120 | 5′ UTR 1 ⁿstem |
| | | (antisense | loop |
| | | strand) | transcriptional |
| | | | fusion to lux |
| JB3 | GGCCAGTGAGCGCGCGTAATAC | pBluescript | To replace 77 |
| | [SEQ ID NO: 10] | KS+ | which formed |
| | | | primer-dimers |
| | | | with GWO43 |
| JB4 | GGGGAGAGTAAAGCTGGCAG | crhC bp 289 | To replace |
| | [SEQ ID NO: 11] | to 269 | GWO43 which |
| | | (antisense | formed primer- |
| | | strand) | dimers with T7 |
| JB5 | CTCTGTTAGGATCCACCATTG | crhC bp −20 | Cloning of crhC |
| | [SEQ ID NO: 12] | to 1 (sense | 5′ UTR and/or |
| | | strand) | ORF behind an |
| | | | E. colt |
| | | | constitutive |
| | | | promoter |
| JB6 | GCGCCAGCTAGCAAATCTCGTC | crhC bp 249 | Cloning of crhC |
| | [SEQ ID NO: 13] | to 228 | 5′ UTR and ORF |
| | | (antisense | behind an E. colt |
| | | strand) | constitutive |
| | | | promoter |
| JB7 | GGACAAGCCGAGATGAG | crhC bp 142 | Shorten cutbacks |
| | [SEQ ID NO: 14] | to 126 | for DNA affinity |
| | | (antisense | chromatography |
| | | strand) | |
| JB8 | GGGGTGCAATCTTCGC | crhC bp −115 | Shorten cutbacks |
| | [SEQ ID NO: 15] | to −100 | for DNA affinity |
| | | (sense | chromatography |
| | | strand) | |
| JB9 | CGTCATTTCCAACTATTA | crhC bp −78 | Shorten cutbacks |
| | [SEQ ID NO: 16] | to −61 | for DNA affinity |
| | | (sense | chromatography |
| | | strand) | |
| JB10 | CCTGTACTCTGTTAAG | crhC bp −26 | Shorten cutbacks |
| | [SEQ ID NO: 17] | to −11 | for DNA affinity |
| | | (sense | chromatography |
| | | strand) | |
| JB11 | GATAGCGAATACCCCAATG | crhC bp 15 | Shorten cutbacks |
| | [SEQ ID NO: 18] | to −4 | for DNA affinity |
| | | (antisense | chromatography |
| | | strand) | |
| JB12 | GGTTTGAGGGGCGGGATG | crhC bp 51 | Shorten cutbacks |
| | [SEQ ID NO: 19] | to 34 | for DNA affinity |
| | | (antisense | chromatography |
| | | strand) | |
| JB13 | CTTAACAGAGTACAGG | crhC bp −11 | Shorten cutbacks |
| | [SEQ ID NO: 20] | to −26 | for DNA affinity |
| | | (antisense | chromatography |
| | | strand) | |
| JB14 | TTCCAACGATTAAGATTA | crhC bp −72 | Mutate AT-rich |
| | [SEQ ID NO: 21] | to −55 | element of crhC |
| | | (sense | promoter |
| | | strand) | |
| JB15 | CCCCAATGGTAGATCTTAACAG | crhC bp 4 | crhC promoter |
| | [SEQ ID NO: 22] | to −18 | only |
| | | (antisense | transcriptional |
| | | strand) | fusion to the lux |
| | | | operon |
| JB18 | CATGAATTCTTTAGGGGCTGGT | crhC bp −156 | crhC AT-rich |
| | [SEQ ID NO: 23] | to −135 | element and −10 |
| | | (sense | region for |
| | | strand) | transcriptional |
| | | | fusion to lux |
| JB19 | CCCGAATTCTGAATCTTAACA | crhC bp 3 | crhC AT-rich |
| | [SEQ ID NO: 24] | to −17 | element and −10 |
| | | (antisense | region for |
| | | strand) | transcriptional |
| | | | fusion to lux |
| JB20 | 5′-GCGAATTCGGATCCAACTATTAATATT | crhC bp −31 | Along with JB21, |
| | AAAGTTTAGAGAAAGGATCCGAATTCGG- | to 25 (sense | encodes the crhC |
| | 3′ | strand) | AT-rich element |
| | [SEQ ID NO: 25] | 3′ | for |
| | | | transcriptional |
| | | | fusion in front of |
| | | | the lux operon |
| JB21 | 5′-CCGAATTCGGATCCTTTCTCTAAACTT | crhC bp 25 | Along with JB20, |
| | TAATATTAATAGTTGGATCCGAATTCGC- | to −31 | encodes for the |
| | 3′ | (antisense | crhC AT-rich |
| | [SEQ ID NO: 26] | strand) | element for |
| | | | transcriptional |
| | | | fusion in front of |
| | | | the lux operon |
| JB22 | GTTTTCCCAGATCTAGTTTGAG | crhC bp 269 | Cloning crhC 5′ |
| | [SEQ ID NO: 27] | to 248 | UTR stem loop |
| | | (antisense | structure(s) b/w a |
| | | strand) | constituitive |
| | | | promoter and lux |
| JB23 | CGCCTAAGACGGATCCCGCTAC | crhC bp 62 | Cloning crhC 5′ |
| | [SEQ ID NO: 28] | to 83 (sense | UTR 2nd stem |
| | | strand) | loop structure b/w |
| | | | a constituitive |
| | | | promoter and lux |
| B*JB12 | GGTTTGAGGGGCGGGATG | crhC bp 50 | Biotinylated |
| | [SEQ ID NO: 29] | to 34 | JB12 for DNA |
| | | (antisense | affinity |
| | | strand) | chromatography |
| pNLPIO- | GCTTCCCAACCTTACCAGAG | pNLP 10 | Sequencing |
| F | [SEQ ID NO: 30] | | pNLP10 |
| | | | constructs |
| pNLPIO- | CACCAAAATTAATGGATTGCAC | pNLP10 | Sequencing |
| R | [SEQ ID NO: 31] | | pNLP10 |
| | | | constructs |
| JSA12 | CGGTAGAGTTGCCCCTACTCCGGTTTTAG | tRNA | Probe for |
| | [SEQ ID NO: 32] | | Northern RNA |
| | | | load control |

*Restriction sites found within the primer are shown in blue (BamH I), red (EcoR I), green (Bgl II), and orange (Nhe 1).

1.3.5 DNA Ligation

Digested DNA fragments, purified from agarose gels, were ligated into digested plasmid vectors with compatible ends. Ligation reactions (20 μL) were performed with 1 unit of T4 DNA ligase (Roche), 1× Ligase buffer (Roche), and various insert to vector ratios. The ligation reaction was incubated at 15° C. or 4° C., for 16-20 hours.

When blunt-end cloning PCR products, a fill-in reaction (Ausubel et al., 1995) was performed with Klenow DNA polymerase (Roche). Blunt-ended (2 pmoles) and symmetrically digested (50 pmoles) vectors were dephosphorylated with calf alkaline phosphatase (CIP) (Roche). 1 unit of CIP and one-tenth volume of the supplied buffer (Roche) were incubated with the appropriate concentration of digested vector for 30 minutes at 37° C. A second unit of CIP was added and incubation continued at 45° C. for 45 minutes. CIP was inactivated by phenol: chloroform extraction and ethanol precipitation method as above (Example 1.3.3) and the dephosphorylated DNA was resuspended in mQdH₂O at a final concentration of 0.1 μg/μL.

1.3.6 Bacterial Transformation

DH5α cells were made chemically competent by treating with rubidium chloride/calcium chloride solutions under cold conditions (Ausubel et al., 1995). An overnight culture of DH5α was diluted 1:100 into 100 mL of fresh LB and grown to an OD₆₀₀ of 0.6 by shaking (200 rpm) at 37° C. for approximately 3 hours. The culture was cooled on ice for 30 minutes and then harvested at 4° C. by centrifugation in a Beckman JA-14 rotor at 4000×g for 10 minutes. Upon decanting the supernatant, the pellet was gently resuspended in one-half volume of ice-cold Buffer A (10 mM RbCl, 10 mM MOPS, pH 7.0) and incubated on ice for 20 minutes. The cells were harvested as above and the pellet resuspended in one-half volume of ice cold Buffer B (10 mM RbCl, 0.1 M MOPS, pH 6.5, 50 mM CaCl₂). The cells were incubated on ice for a minimum of 30 minutes, pelleted, and resuspended in one-tenth volume of ice-cold Buffer B. DMSO was added to a final concentration of 7% and the competent cells were dispensed in 600 μL aliquots. The competent cells were flash frozen in liquid nitrogen and stored at −80° C.

When transforming the chemically competent DH5α cells with foreign DNA, the heat shock method was employed, with a few modifications (Ausubel et al., 1995). Competent cells were thawed on ice and for each transformation, 200 μL of competent DH5α cells were used. The amount of ligated DNA added to the competent cells depended on the plasmid copy number and concentration of DNA used in the ligation reaction. For high copy plasmids and/or DNA concentrations greater than 1 μg, half of the ligation reaction (10 μL) was added. For low copy plasmids and/or DNA concentrations less than 1 μg, the full ligation reaction (20 μL) was mixed with the 200 μL of competent cells. The transformation mixture was placed on ice for 30 minutes, heat shocked at 42° C. for 2 minutes, and incubated on ice for 5 minutes. Prewarmed LB medium (1 mL) was added to each transformation and incubated at 37° C. for 1-2 hours. For cells transformed with high copy plasmids, 100 μL of the transformation was plated on LB plates containing the appropriate antibiotics and incubated at 37° C. overnight. For low copy plasmids, the complete transformation was harvested by microcentrifugation and the supernatant decanted. The pellet was resuspended in the remaining 50-100 μL of medium and plated on selective LB plates. For detection of β-galactosidase activity using blue/white selection, 50 μL of a 5:1 X-gal: IPTG (2% [w/v] X-gal: 100 mM IPTG) mixture was overlaid on the LB plates 30 minutes prior to plating the transformed cells.

1.3.7 Bacterial Electroporation

To increase the efficiency of transformation, electrocompetent DH5α cells were prepared through a series of ice-cold mQdH₂O and glycerol washes (Ausubel et al., 1995). An overnight culture of DH5α cells was diluted 1:100 into 25 mL of fresh LB and grown to an OD₆₀₀ between 0.5-0.7 at 37° C., with shaking at 200 rpm is OK. The cells were chilled for 15 minutes in an ice water bath and harvested by centrifuging at 4200×g for 10 minutes at 4° C. The cell pellet was gently washed three times in 25 mL of ice-cold sterile mQdH₂O, and three times in 10% [v/v] ice-cold glycerol, with centrifugation at 4200×g for 10 minutes between each wash. The final resuspension was dispensed in 50 μL aliquots, flash frozen in liquid nitrogen, and stored at −80° C. For each electroporation, 50 μL of electrocompetent cells were thawed on ice, mixed with 100 ng of DNA or 1 μL of unknown DNA concentration, and transferred to a chilled, 1 mm gap sterile electroporation cuvette (Molecular BioProducts). The mixture was electroporated in an Eppendorf Electroporator 2510, pulsed with 1800 volts at a time constant less then 5 msec. Immediately after electroporation, 300 μL of LB medium was added and the mixture incubated at 37° C. for 1.5 hours without shaking. Appropriate volumes of transformed cells were plated on selective nutrient agar plates, using the same criteria mentioned in Example 1.3.6.

1.3.8 DNA Sequencing

Dideoxy chain termination sequencing was performed using the DYEnamic ET Terminator Cycle Sequencing Kit (Amersham Biosciences). Prior to sequencing, small-scale plasmid preparations were treated with 1 μL of RNase A (10 mg/mL) and incubated at 37° C. for 15 minutes. RNase A was inactivated by extracting with phenol:chloroform and ethanol precipitation (Example 1.3.3) and the DNA was resuspended in sterile mQdH₂O. In a final volume of 20 μL, the sequencing reaction contained 1× sequencing dilution buffer (80 mM Tris, 2 mM MgCl₂, pH 9.0), 1× sequencing reagent mix (Thermo sequenase II DNA polymerase, ddNTPs, dNTPs, 80 mM Tris, 2 mM MgCl₂, pH 9.0), 5 pmoles of primer, and 500-800 ng of template DNA. The reaction was amplified in a thermocycler (MJ Research) using the following parameters: 95° C. denature for 30 sec, 50° C. annealing for 15 sec, 60° C. elongation for 60 sec, cycled 25 times. Once cycling was complete, the reaction was precipitated with 4 volumes of 95% [v/v]ethanol and one-tenth volume of sodium acetate/EDTA buffer (150 mM sodium acetate, pH 8.0, 225 mM EDTA). The reaction was briefly mixed by vortexing and incubated for 15 minutes at 4° C. The precipitate was pelleted by microcentrifugation at room temperature for 15 minutes at 14,000×g×g. The pellet was washed with 400 μL of 70% [v/v]ethanol, microcentrifuged for 5 minutes, and air-dried. The dried DNA pellet was submitted to the Molecular Biology Service Unit (University of Alberta, Edmonton, Alberta) for automated sequencing using a Genetic Analyzer 3100 (prior to September 2004) or an Applied Bioscience 377 (after September 2004).

1.3.9 Radioactive Labelling of DNA

1.3.9.1 End-labeling DNA

The 5′ ends of oligonucleotides or linear dsDNA were radioactively labeled with [Y-³²P]dATP (Perkin Elmer) and polynucleotide kinase (PNK) (NEB). Each end-labeling reaction contained 5 pmoles of target DNA, 1×PNK Buffer (NEB), 50 μCi of [γ-³²P]-dATP, and 0.01 units of PNK. The 20 μL reaction was incubated in a 37° C. water bath for 30 minutes, chilled on ice, and the PNK inactivated by heating for 10 minutes at 75° C. and/or by the addition of 8 μL of 3% [w/v] Dextran Blue (Sigma) in TE (10 mM Tris-HCl, pH8, 1 mM EDTA, pH 8). Unincorporated nucleotides were removed by passage through either a 200-400 mesh Biogel-P2 column for oligonucleotides (Bio-Rad) or a Sephadex G-50 column for DNA fragments >40 bp (Amersham Pharmacia Biotech), prepared in a 1 mL syringe. The probe was eluted with TE buffer and the blue fraction collected into a sterile microfuge tube. The specific activity of the purified probe (1 μL) was determined by Cerenkov counting in a Beckman LS 3801 scintillation counter.

1.3.9.2 Random-Primer Labeling of DNA

Linear dsDNA was radioactively labeled using the random primer labeling technique (Feinberg and Vogelstein, 1983). Digested (Example 1.3.2) and gel purified (Example 1.3.3) dsDNA fragments were denatured by boiling for 5 minutes and immediately chilled on ice for 5 minutes. Reagents were added in the following order to synthesize a radioactively labeled complementary strand; 25-100 ng of denatured dsDNA, 75 μM of each dNTP (A, G, and T), 1× hexanucleotide buffer (Roche), 25 μCi of [α-³²P]dCTP (Perkin Elmer), and 1 unit of Klenow (Roche), in a 10 μL final volume. The reaction was incubated for 1 hour in a 37° C. water bath and quenched by the addition of 3% [w/v] Dextran Blue (Sigma) in TE (10 μL). Unincorporated nucleotides were removed using a Sephadex G-50 column and the specific activity determined by Cerenkov counting (Example 1.3.9.1).

1.3.10 Promoter Nested Deletion Construction

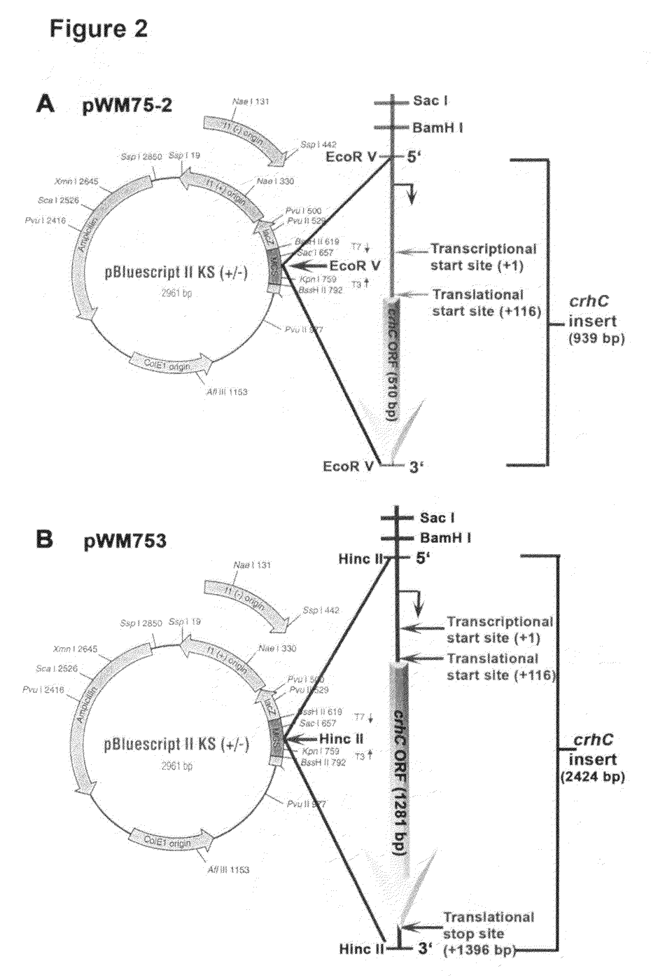

Nested deletion constructs within the crhC promoter were created (DC, unpublished data, and JW, Ward, 2001) using the plasmids pWM753 and pWM75-2, respectively (FIG. 2, Table 3). pWM75-2 contains a 939 bp EcoR V insert containing the complete crhC promoter (315 bp), 5′ UTR (115 bp) and 510 bp of the 1275 bp crhC ORF, cloned into the EcoR V site of pBluescript KS+(Stratagene) (FIG. 2A). pWM753 contains a 2424 bp Hinc II insert containing the full-length crhC gene (promoter, 5′ UTR, ORF, and the 257 bp 3′UTR) cloned into pBluescript KS+(FIG. 2B).

TABLE 3
crhC Promoter Deletion Plasmid Constructs

| | Parent | Insert | Cutback | Motifs |
|---|---|---|---|---|
| Plasmid | Plasmid | Size | Start Site | Removed |
| JW2 | pWM75-2 | 892 bp | crhC bp −264 | none |
| JW3 | pWM75-2 | 824 bp | crhC bp −196 | none |
| JW4 | pWM75-2 | 741 bp | crhC bp −113 | none |
| JW5 | pWM75-2 | 664 bp | crhC bp −36 | AT-rich element |
| JW6 | pWM75-2 | 649 bp | crhC bp −21 | AT-rich element |
| JW7 | pWM75-2 | 608 bp | crhC bp +20 | Complete crhC promoter |
| JW8 | pWM75-2 | 520 bp | crhC bp +108 | Complete crhC promoter |
| | | | | and cold shock box |
| DC1 | pWM753 | 2227 by | crhC by −574 | none |
| DC2 | pWM753 | 1864 by | crhC by −211 | none |
| DC3 | pWM753 | 1845 bp | crhC bp −192 | none |
| DC4 | pWM753 | 1729 bp | crhC bp −76 | none |
| DC5 | pWM753 | 1696 bp | crhC bp −43 | AT-rich element |
| DC6 | pWM753 | 1656 bp | crhC bp −3 | AT-rich element and −10 |
| | | | | region |
| DC7 | pWM753 | 1484 bp | crhC bp +170 | Complete crhC promoter, |
| | | | | 5′UTR, and downstream |
| | | | | box |
| DC8 | pWM753 | 1416 bp | crhC bp +238 | Complete crhC promoter, |
| | | | | 5′UTR, and downstream |
| | | | | box |

Exonuclease III (Exo III) digestion was performed on BamH I/Sac I digested pWM75-2 and pWM753 (Table 3) using an Erase-a-base kit (Promega). Details of the selected deletions are presented in Table 3. 3′ to 5′ exonuclease activity was stopped at one-minute intervals, for a total of ten minutes, and the single-stranded DNA degraded with an S1 nuclease mix (Promega). The samples were heat inactivated at 70° C. for 10 minutes, filled in with Klenow (Roche) (Example 1.3.5), blunt-end religated, and transformed into E. coli DH5α (Example 1.3.6). The identification of each deletion was confirmed by PCR using the primer pairs T7:GWO43 or JB3:JB4 (Table 2) and by sequencing (Example 1.3.8).

1.4 Protein Manipulation

1.4.1 Protein Isolation

1.4.1.1 Protein Extraction from Cyanobacteria

From a BG-11 plate, a heavy inoculum of cyanobacteria was aseptically inoculated into 50 mL BG-11 and grown at 30° C. with shaking until exponential phase (3 days) (Example 1.1). The complete 50 mL culture was transferred to 300 mL BG-11 and grown at 30° C. with aeration until exponential phase (4 days). When required, the 300 mL culture was aliquoted into two 150 mL cultures and placed either at 30° C. (optimal) or at 20° C. (cold shock/stress), for the indicated times. The cells were harvested at the appropriate temperature by centrifugation (Janetzki T5) in 15 mL polypropylene tubes (Corning) at 6,000 g for 10 minutes. The pellet was washed with an equal volume of 50/100 TE (50 mM Tris, 100 mM EDTA) and 1 mM DTT, and recentrifuged. Upon decanting the supernatant, the cell pellet was flash frozen in liquid nitrogen, and either stored at −80° C. or thawed immediately for protein extraction.

For cell lysis, the pellet was resuspended in an equal volume of cyanobacterial protein extraction buffer (20 mM Tris-HCl, pH 8, 10 mM NaCl, 1 mM EDTA, pH 8) containing a protease inhibitor cocktail (Complete Mini-Roche) and lysed at a temperature corresponding to the growth temperature. Cell lysis was accomplished by vortexing in the presence of an equal volume of Dyno-Mill Lead free 0.2-0.3 mm glass beads (Impandex Inc.) for 8×1 minute, with one-minute incubation in an ice-water bath between each vortex. Cellular debris was removed by microcentrifugation for 5 minutes at 14,000×g. Proteins were quantified using the Bradford Assay (Bio-Rad), with BSA as the standard. Protein aliquots were stored in 10% [v/v] glycerol at −80° C.

1.4.1.2 Protein Extraction from E. coli

An overnight E. coli culture was diluted 1:50 into 20 mL of fresh LB and incubated with shaking at 37° C. until an OD₆₀₀ of 0.6. For cold-shocked cells, 10 mL of the exponentially grown culture was transferred to a 20° C. water bath shaker (Gyrotory Model G76, New Brunswick Scientific) for the indicated times. Cells were harvested by microcentrifuging at 14,000×g at the appropriate temperature for 5 minutes, flash froze, and the pellets stored at −80° C. Thawed cells were resuspended in 500 μL of chilled cyanobacterial extraction buffer including a protein inhibitor cocktail (Example 1.4.1.1) and lysed by sonicating 4×30 seconds with 1 minute in an ice-water bath, between each sonication. Cellular debris was removed by microcentrifugation and protein concentration quantified by the Bradford assay, as described above (Example 1.4.1.1).

1.4.2 Protein Electrophoresis

1.4.2.1 Non-Denaturing Polyacrylamide Gel Electrophoresis (PAGE)

EMSA reactions (Example 1.5.1) were separated on 8%, 10%, or 12% [w/v] native 1×TBE or 1×TAE gels. Each 12% native gel contained 1875 μL of 40% [w/v] acrylamide:bis(37.5:1) (BioRad), 78.12 μL of 1×TBE (0.1 M Tris, 0.1 M Boric acid, 2 mM EDTA, pH 8) or 1×TAE (40 mM Tris-acetate, 1 mM EDTA, pH 8), 93.75 μL of 100% glycerol, 3184.4 μL of mQdH₂O, 311.5 μL of 1.5% [w/v] APS, and 3.125 μL of TEMED. Slab gels (5 mL) were cast in a Bio-Rad Mini-PROTEAN II electrophoresis cell, allowed to polymerize for 30 minutes, and the wells rinsed with running buffer. Prior to loading, the gel was electrophoresed at 150 V for 30 minutes in 1× running buffer corresponding to the buffer used to make the gel. Once the samples were loaded, the gel was electrophoresed at a constant voltage of 150 V for 60 minutes at 37° C. After electrophoresis, the gel was dried at 70° C. for 45 minutes (Savant Slab Gel Dryer SGD4050) and exposed on X-ray film at −80° C. or on a phosphoimager (Molecular Dynamics), for visualization.

1.4.2.2 SDS-Polyacrylamide Gel Electrophoresis (SDS-PAGE)

Protein samples were denatured and separated on a 10% [w/v] SDS-PAGE gel cast in a Bio-Rad Mini-PROTEAN II electrophoresis cell. The resolving gel was comprised of: 937.5 μL of 40% [w/v] acrylamide:bis(37.5:1)(BioRad), 468.75 μL of 3 M Tris-HCl, pH 8.8, 37.5 μL of 10% [w/v] SDS, 2,120 μL of mQdH₂O, 187.5 μL of 1.5% [w/v] APS, and 1.875 μL of TEMED. A 3.75 mL aliquot of resolving gel was poured into the cast and immediately overlaid with isopropanol and allowed to polymerize for 30 minutes. The isopropanol was thoroughly rinsed off with mQdH₂O and dried with 3 MM Whatmann paper. The stacking gel was comprised of: 125 μL of 40% [w/v] acrylamide:bis(37.5:1), 315 μL of 0.5 M Tris, pH 6.8, 11.5 μL of 10% [w/v] SDS, 741.25 μL of mQdH₂O, 50 μL of 1.5% [w/v] APS, and 1.25 μL of TEMED. A 1.25 mL aliquot of the stacking gel was overlaid on the resolving gel, a 10-well comb inserted, and allowed to polymerize for 30 minutes.

One-third volume of SDS loading buffer (125 mM Tris, pH 6.8, 4% [w/v] SDS, 20% [v/v] glycerol, 10% [v/v] α-mercaptoethanol, and 0.02% bromophenol blue) was added to each protein sample prior to incubation in a boiling water bath for 5 minutes. Samples were electrophoresed in 1×SDS running buffer (25 mM Tris, 0.192 M glycine, 0.1% [w/v] SDS) at a constant voltage of 200 V for 1-1.5 hours. Protein sizes were determined by electrophoresing 10 μL of a 1:20 dilution of Low Range (LR) SDS-PAGE standards (Bio-Rad) or 10 μL of the Broad Range (BR) Prestained Protein markers (NEB), alongside the protein samples.

1.4.3 Staining SDS-Polyacrylamide Gels

Following electrophoresis, proteins were fixed in the gel by shaking for 5 minutes in destain solution (30% [v/v] methanol, 10% [v/v] glacial acetic acid). Proteins were visualized by staining with Coomassie Brilliant Blue Stain (14% [v/v] methanol, 10% [v/v] glacial acetic acid, 0.25% [w/v] Coomassie Brilliant Blue R250) for 10 minutes with shaking. Gels were destained until the desired stain intensity and then dried onto 3 MM filter paper at 70° C. for 45 minutes (Example 1.4.1.2).

PAGE gels containing proteins for protein sequencing were stained with SeeBand Protein Staining Solution (Gene Bio-Application) or Silver Stain Plus (Bio-Rad). SeeBand Protein Staining Solution was used according to the manufacturer's instruction. The Silver staining protocol had a few modifications. Following fixation, the staining solution was mixed in the followed order; 8.75 mL mQdH₂O, 1.25 mL of Silver Complex solution, 1.25 mL of Reduction Moderator solution, and 1.25 mL Image Development reagent, poured over the gel with vigorous shaking. 25 mL of Accelerator solution was quickly added and the gel was stained until the optimal banding intensity. The reaction was stopped and stored in 5% [w/w]acetic acid and when necessary, the desired polypeptide excised from the gel and sent to the Institute for Biomolecular Design (IBD) (University of Alberta, Edmonton, AB, Canada) for protein identification. Automated in-gel tryptic digestion was performed and the tryptic peptides subjected to LC/MS/MS. The generated LC/MS/MS data were used as queries for database searches using Mascot (Matrix Science, UK) and the National Center for Biotechnology Information (NCBI).

1.4.4 Protein Precipitation

Proteins eluted from DNA affinity chromatography columns (Example 1.5.2) were concentrated by TCA precipitation. Protein samples were mixed with one-tenth volume of 100% trichloro-acetic acid (TCA) (Anachemia) and one-tenth volume deoxycholate (10 mg/mL) (Sigma) and incubated on ice for 30 minutes. The protein precipitate was pelleted at 14,000×g for 15 minutes at 4° C. and washed 4-5 times with 1 mL of 100% chilled acetone. The protein pellet was resuspended in 20 μL of 0.1 M DTT, 0.1 M Na₂CO₃ and size fractionated by SDS-PAGE gel electrophoresis (Example 1.4.1.2).

1.4.5 Western Analysis

Equivalent concentrations of protein extracts (Example 1.4.1.2), as determined by the Bradford assay (Example 1.4.1.1), were loaded and separated on a 10% [w/v] SDS-PAGE gel (Example 1.4.1.2). Following electrophoresis, the proteins were immobilized to a solid matrix through electroblotting, using the semi-dry transfer method. Proteins from an unstained SDS-PAGE gel were transferred to a 0.45 micron Hybond ECL nitrocellulose membrane (Amersham Pharmacia Biotech) using an Electrophoretic Transfer System ET-10 (Tyler Research Instruments), according to the manufacturer's instructions. Prior to transfer, the membrane and four pieces of 3 MM Whatmann paper were soaked in 1× Tyler transfer buffer (25 mM Tris, 150 mM glycine, pH 8.3, 20% [v/v] methanol) for 30 minutes and the SDS-PAGE gel soaked for 5 minutes. The transfer components were assembled in the following order: 2 pieces of 3 MM Whatmann paper, SDS-PAGE gel closest to the cathode, membrane, and 2 pieces of 3 MM Whatmann paper. One gel was transferred at room temperature for 60 minutes at a constant 60 mA, whereas two gels were transferred at a constant 80 mA for 60 minutes. Following transfer, the gel was stained with Coomassie Brilliant Blue (Example 1.4.1.3) to determine the efficiency of protein transfer.

Western blot analysis was performed as described by Chamot et al., 1999

The nitrocellulose membrane was blocked by incubating at room temperature in fresh 1×BLOTTO (1×TBS (150 mM NaCl, 10 mM Tris-HCl, pH 8.0), 5% [w/v] skim milk powder (Carnation), 0.02% [v/v] sodium azide) for 30 minutes, with gentle agitation. To fresh 1×BLOTTO, anti-crhC serum (1:5000) (Chamot et al., 1999; Yu, 1999) was added and incubated at room temperature for 16-24 hours. Three consecutive 10 mL washes were performed for 10 minutes with 1×TBS, 1×TBST (0.05% [v/v] Tween in 1×TBS), and 1×TBS, to reduce background. The membrane was incubated for 30 minutes in 20 mL 1×TBS containing goat anti-rabbit IgG antibody conjugated to horse-radish peroxidase (HRP) (1:20,000) (Sigma). The membrane was consecutively washed for 10 minutes with 10 mL of 1×TBS, 1×TBST, and 1×TBS and wrapped in saran warp. CrhC was visualized using the ECL Western Blotting Detection kit (Amersham Biosciences), according to the manufacture's instructions, and the resulting chemilluminescence detected by autoradiography.

1.5 Protein—DNA Interactions

1.5.1 Electrophoretic Gel Mobility Shift Assays (EMSA)

Promoter target DNA fragments were generated by PCR (Table 2) (Example 1.3.4) and/or by RE digestion of the appropriate vector (Table 4 & 5). All DNA targets were gel purified as described above (Example 1.3.3), radioactively labeled by end-labeling (Example 1.3.9.1), and the specific activity determined by Cerenkov counting. For EMSA analysis, the probe was diluted in mQdH₂O to a specific activity of 2000-5000 cpm/μL. Protein—DNA interactions were carried out in 20 μL reactions comprised of, 2000-5000 cpm of target DNA, 1×EMSA buffer (10 mM Tris, pH 7.5, 50 mM NaCl, 1 mM EDTA, 5% [v/v] glycerol, 1 mM DTT, 10 mM MgCl₂), 1 μg of poly dI/dC (Roche), and the indicated amounts of Anabaena protein extract (Example 1.4.1.1). The reaction was incubated at the indicated temperature (4° C., 20° C., 37° C.) for 30 minutes prior to loading on a 8%, 10%, or 12% native 1×TAE or 1×TBE polyacrylamide gel (Example 1.4.1.1). Alongside the samples, 5 μL of 5×DNA loading buffer (Example 1.3.2) was also loaded to track the approximate migration distance of the DNA, as no loading dye was added directly to the samples. The gels were electrophoresed at 150 V for 60 minutes, dried, and visualized by autoradiography at −80° C.

TABLE 4
Parent Plasmids

| | | Selective | Importance/Insert | |
|---|---|---|---|---|
| Plasmid | Source | Marker | sequence | Use |
| pBluescript | Stratagene | Ampicillin | Cloning vector | Cloning |
| KS+ | | | | |
| (pBS) | | | | |
| pWM753 | Magee, | Ampicillin | The complete 2424 bp | Promoter deletion |
| | 1997 | | crhC gene (promoter, | construction/ |
| | | | ORF, 5′ and | Cloning/ |
| | | | 3′ UTR) cloned into the | Sequencing/ |
| | | | Hinc II site of pBS (FIG. | crhC expression |
| | | | 2B) | analysis |
| pWM75-2 | Magee, | Ampicillin | A 939 bp crhC insert | Promoter deletion |
| | 1997 | | (promoter, 5′ UTR, and | construction/ |
| | | | portion of | Cloning |
| | | | ORF) cloned into the EcoR | Sequencing/ |
| | | | V site of pBS (FIG. 2A) | EMSA analysis |
| pCS26 | Dr. Mike | Kanamycin | Contains luxCDABE | Cloning/crhC |
| | Surrette | | operon for transcriptional | promoter motif |
| | | | reporter fusion construct | luciferase |
| | | | generation | assays in E. coli |
| | Dr. Tracy | Kanamycin | Contains luxCDABE | Cloning/crhC |
| | Ravio | | operon for transcriptional | promoter and 5′ |
| | | | reporter fusion construct | UTR |
| | | | generation | luciferase assays |
| | | | | in E. coil |
| | Dr. Mike | Kanamycin | pSC26 containing the | Post-transcription |
| | Surrette | | strong SIG16 constitutive | studies/ |
| | | | E. coli | 5′ UTR luciferase |
| | | | promoter in front of the lux | assays in E. coli |
| | | | operon | |
| | Dr. Mike | Kanamycin | pCS26 containing the | Post-transcription |
| | Surrette | | medium strength SIG11 | studies/ |
| | | | constitutive | 5′ UTR luciferase |
| | | | E. coil promoter in front of | assays in E. coli |
| | | | the lux operon | |

TABLE 5
Plasmid Constructions

| | | | | | Cloning Strategy | |
|---|---|---|---|---|---|---|
| | Parent | Selective | Insert | Region of crhC | (vecot digest/ | Molecular |
| Plasmid | Plasmid | Marker | Size | inserted | insert digest) | Line |
| pWM753R | pWM753 | Ampicillin | 2424 bp | Reverse | Hinc II digest and | Construction |
| | | | | orientation of full | religation of | of pJBm1 |
| | | | | length crhC gene | pWM753 | and pJBm2 |
| | | | | from pWM753 | | |
| pJBp1 | pCS26 | Kanamycin | 39 bp | AT-rich element | BamH IBamH I | Luciferase |
| | | | | of the | with annealed | Assay/ |
| | | | | crhCpromoter | complementary | Promoter |
| | | | | | primers | studies |
| | | | | | (JB20:JB21) | |
| | | | | | (Example 2.7) | |
| pJBp2 | pNLP10 | Kanamycin | 154 bp | AT-rich element | EcoR I/EcoR I | Luciferase |
| | | | | and -10 regions of | with JB18:JB19 | Assay/ |
| | | | | the | PCR product | Promoter |
| | | | | crhC promoter | (Example 2.7) | studies |
| pJBp3 | pNLP10 | Kanamycin | 371 bp | crhC promoter | BamH I/Bgl II | Luciferase |
| | | | | lacking the | with JB1:JBI5 | Assay/ |
| | | | | transcriptional | PCR product | Promoter |
| | | | | start site | (Example 2.7) | studies |
| pJBp4 | pNLP10 | Kanamycin | 511 bp | full-length crhC | BamH I/Bgl II | Luciferase |
| | | | | promoter and the | with JBI:JB2 | Assay/ |
| | | | | 1″ stem loop | PCR product | Promoter |
| | | | | structure of the 5′ | (Example 2.7) | studies |
| | | | | UTR (11 bp into | | |
| | | | | ORF) | | |
| pSIG16 | pSIG16 | Kanamycin | N/A | N/A | Not I digestion to | Construction |
| (lux⁻) | | | | | remove the lux | of pJBm1 |
| | | | | | operon | |
| pSIG11 | pSIGI 1 | Kanamycin | N/A | N/A | Not I digestion to | Construction |
| (lux⁻) | | | | | remove the lux | of pJBm2 |
| | | | | | operon | |
| pJBm1 | pSIG16 | Kanamycin | 1664 bp | full-length crhC 5′ | BamH I/BamH I | Post- |
| | (lux) | | | UTR (both stem | triple ligation with | transcription |
| | | | | loop | PCR (JB5:JB6), | studies |
| | | | | structures), ORF | pWM753R, and | |
| | | | | and 3′UTR | pSIG16(lux) | |
| | | | | | (Example 2.7) | |
| pJBm2 | pSIGI1 | Kanamycin | 1664 bp | full-length crhC 5′ | BamH 1/BamH I | Post- |
| | (lux) | | | UTR (both stem | triple ligation with | transcription |
| | | | | loop | PCR (JB5:JB6), | studies |
| | | | | structures), ORF | pWM73R, and | |
| | | | | and 3′UTR | pSIGI 1(lux) | |
| | | | | | (Example 2.7) | |
| pJBm3 | pSIG1 1 | Kanamycin | 274 bp | both stem loop | BamH I/Bgl II | Luciferase |
| | | | | structures of the | with JB5:JB22 | Assay/ |
| | | | | crhC 5′UTR | PCR product | mRNA |
| | | | | | | stability |
| | | | | | | studies |
| pJBm4 | pSIG1 1 | Kanamycin | 191 bp | 2nd stem loop | BamH I/Bgl II | Luciferase |
| | | | | structure only of | with JB23:JB22 | Assay/ |
| | | | | the crhC 5′UTR | PCR product | mRNA |
| | | | | | | stability |
| | | | | | | studies |
| pJBm5 | pNLP10 | Kanamycin | 2426 bp | full-length crhC | Xho 1BamH I of | mRNA |
| | | | | gene from | pWM753R into | stability |
| | | | | pWM753 | Xho IBamH I | control |
| | | | | | pNLP10 (Example | |
| | | | | | 2.7) | |
| pJBm6 | pNLP10 | Kanamycin | 2168 bp | crhCgene lacking | Hinc II/SnaB I | 3′UTR's |
| | | | | the 3′UTR | pWM753 into | role in |
| | | | | | pBS, Xho I/BamH I | mRNA |
| | | | | | pBS into Xho | stability |
| | | | | | IBamH I pNLP10 | |
| | | | | | (Example 2.7) | |

1.5.1.1 Competition Assays EMSA competition assays were performed using the standard EMSA conditions (Example 1.5.1) with the addition of either competitive or non-competitive DNA. Unlabeled target DNA was used as competitor DNA and an unrelated and unlabeled fragment of DNA of equivalent size was used as the non-competitor DNA. The non-competitor DNA was amplified from within the crhC ORF, using the primers GWO36: WCM1 (Table 2) to produce a 113 bp product.

1.5.1.2 Dephosphorylation Studies

Dephosphorylation studies were performed using calf intestinal alkaline phosphatase (CIP) (Roche), as described by Ausubel et al. (1995). Anabaena protein extract (10 μg) was dephosphorylated prior to the addition of target DNA to the EMSA binding reaction. In a 20 μL final volume, 30 μg of protein extract was incubated with 1×EMSA buffer (Example 1.5.1), 1 μg of poly dI/dC (Roche), at 30° C. for 10 minutes. CIP (1.5 U) and 1 mM ZnSO₄ were added to the mixture and incubation continued at 30° C. for 15 minutes. The enzymatic reaction was terminated by the addition of sodium pyrophosphate (10 mM). The dephosphorylated protein sample was used directly in the EMSA reaction, as described above (Example 1.5.1).

1.5.2 DNA-Binding Protein Purification

DNA affinity chromatography was performed using a μMACS Streptavidin kit (Miltenyi Biotec) and MACS separation columns (Miltenyi Biotec) to isolate and purify proteins that interact with specific DNA targets. Using a biotinylated primer (Table 2), target DNA fragments were PCR amplified (Example 1.3.4) to carry a single biotin tag at the 5′ end, which are specifically bound by target protein(s) via incubation with a total protein lysate. The protein bound biotinylated target DNA is then magnetically labeled by complexing the biotin tag to streptavidin ligands, which are conjugated to paramagnetic Microbeads. This molecular complex is immobilized on μ columns placed in a strong magnetic field generated by a μMAC separator. Following magnetic separation, the bound proteins can be eluted with increasing salt concentration.

In a final volume of 3 ml, protein—DNA interactions were allowed to occur by incubating 10-15 mg of Anabaena protein lysate (Example 1.4.1.1), 1×JB₅₀ buffer ((1×JB buffer=10 mM HEPES, pH 8, 1 mM EDTA, 5% [v/v] glycerol, 1 mM DTT, 10 mM MgCl₂) 1×JB₅₀ buffer=JB buffer+50 mM KCl), 50 μg of poly dI/dC (Roche), and 20-50 μg of biotinylated target DNA, at 4° C. for 2 hours with shaking. Following incubation, 100 μL of μMACS Microbeads conjugated to streptavidin (Miltenyi Biotec) were added and incubated at 4° C. for 30 minutes, to allow for streptavidin-biotin complexes to form. At 4° C., the MACS μ columns (Miltenyi Biotec) were placed in the magnetic field of the μMACS separator (Miltenyi Biotec) and equilibrated by washing sequentially with 300 μL of Protein Application Equilibration Buffer (Miltenyi Biotec) and 300 μL of 1×JB₅₀ buffer. The 3 mL binding reaction was added to the column in 500 μL aliquots and the flow-through collected. The column was stringently washed five times with 250 μL of 1×JB₅₀ (W1), three times with 250 μL of 1×JB₁₀₀ (W2) (1×JB buffer+100 mM KCl), three times with 250 μL of 1×JB₂₅₀ (W3) (1×JB buffer+250 mM KCl), three times with 250 μL 1×JB₁₀₀₀ washes (W4) (1×JB buffer+1 M KCl), and three times with 250 μL of 1×JB₂₀₀₀ (W5) (1×JB buffer+2 M KCl). The column was completely cleaned of proteins by rinsing twice with 250 μL of 1×JB₂₀₀₀, with the column removed from the magnet. The eluted fractions (W1-W5) were TCA precipitated (Example 1.4.3), separated on a 10% SDS-PAGE gel (Example 1.4.1.2), and polypeptides of interest excised from the gel and provided to IBD for protein sequencing (Example 1.4.1.3).

1.6 RNA Manipulation

1.6.1 RNA Extraction from E. coli

An overnight E. coli culture was diluted 1:25 into 25 mL of fresh LB (including appropriate antibiotics) and incubated at 37° C. until an OD₆₀₀ of 0.6. Half of the culture was removed into 50 mL sterile flasks and cold-shocked at 20° C. with shaking at 200 rpm for the indicated times. Optimally grown samples remained at 37° C. where they were harvested by centrifugation (Janetzki T5) at 4,000×g in 15 mL polypropylene tubes. At 37° C. or 20° C. and under nuclease free conditions, the cell pellet was resuspended in 650 μL of 65° C. RNA extraction buffer (1% [w/v] SDS, 10 mM sodium acetate, pH 4.5, 150 mM sucrose) and transferred to a clean microfuge tube. 650 μL of hot (65° C.) phenol was added and the mixture extracted at 65° C. for 15 minutes. After microcentrifugation at 7000×g for 5 minutes, proteins were extracted with equal volume organic phases; once with phenol, twice with phenol:chloroform (1:1), and once with chloroform:isoamyl alcohol (24:1), with a 5 minute microcentrifugation at 7000×g, between each extraction. The RNA was precipitated by adding an equal volume of 4 M LiCl to the extracted aqueous phase, mixing well and incubating at −20° C. overnight. Precipitated material was pelleted at 14,000×g for 30 minutes at 4° C., resuspended in 300 μL of nuclease-free TE (10 mM Tris-HCl, pH 8, 1 mM EDTA, pH 8), and precipitated by the addition of one-tenth volume 3M sodium acetate, pH 5.2 and 1.5× volume of 100% ethanol and stored at −80° C. When the RNA was needed, the sample was microcentrifuged at 14,000×g for 15 minutes at 4° C. and washed with of 80% [v/v]ethanol (1 mL). The air-dried pellet was resuspended in RNase-free sterile mQdH₂O (30-50 μL) and quantified spectrophotometrically at a wavelength of 260 nm. The RNA concentration was determined spectrophotometrically similar to that of DNA (Example 1.3.1) using an extinction coefficient of 40 μg/mL (1.0 A₂₆₀ nm=40 μg/mL dsRNA).

1.6.2 Northern Analysis

Northern analysis was carried out as described by Ausubel et al. (1995). In a nuclease-free environment, 10-20 μg of RNA (Example 1.6.1) was denatured and electrophoresed on a ˜100 mL formaldehyde gel (1.2% [w/v] agarose, 97 mL of 1×MOPS Buffer, 5.1 mL of formaldehyde). Prior to electrophoresis, RNA samples were denatured by heating at 65° C. for 15 minutes in 1×RNA formaldehyde loading buffer (50% [v/v] formamide, 17% [v/v] formaldehyde, 7% [v/v] glycerol, 0.2% [w/v] bromophenol blue in 1×MOPS), briefly chilled on ice, and ethidium bromide (1 μl of 0.5%) added. RNA samples were separated on a 5.1% formaldehyde, 1.2% agarose gel in 1×MOPS running buffer (20 mM MOPS, 5 mM sodium acetate, 1 mM EDTA, pH 7), at 125 V for 2-3 hours, with gel rotation every 30 minutes. Monitoring of RNA degradation and loading consistency were visualized using a Syngene Genius Bio Imaging System.

RNA was transferred from the agarose gel to a positively charged Hybond-XL nylon membrane (Amersham Pharmacia Biotech) using capillary action and 20×SSC (3 M NaCl, 0.3 M Na₃citrate, pH 7). The high salt upward capillary transfer was performed according to standard procedures (Ausubel et al., 1995), with transfer occurring over 16-24 hours. Following dismantling of the transfer apparatus, the ribosomal RNA was distinctly marked and the RNA was immobilized by UV-crosslinking at 120 mJ/cm² in a SpectroLinker XL-1000 UV Crosslinker (Spectronics Corporation). Excess salts were removed by washing at 65° C. for 60 minutes in a Post-bake wash buffer (1×SSC, 0.1% [w/v] SDS) and transferred to a seal-a-meal bag (Rival). To reduce background, the membrane was blocked by prehybridization at 65° C. in 1 mL/cm² of membrane in Aqueous Hybond solution (50% [v/v] formamide, 5×Denhardts, 0.2% [w/v] SDS, 5×SSPE, and 50 μg/mL of boiled salmon sperm ssDNA) for 4-24 hours. 1×10⁶ cpm/mL of random-primed DNA probe (Example 1.3.9.2) was added to fresh Aqueous Hybond (1 mL/cm²) solution and incubation continued at 65° C. for 16-24 hours. Following hybridization, the membrane was washed once at 65° C. with a low stringency wash (1×SSPE [180 mM NaCl, 10 mM NaH₂PO₄, 1 mM EDTA, pH 8], 0.1% [w/v] SDS) and once at 65° C. with a high stringency wash (0.1×SSPE, 0.1% [w/v] SDS). The membrane was wrapped in saran wrap and the bound probe was detected by autoradiography at −80° C. for 24-48 hours. Prior to storage, the membrane was stripped of probe by immersion in 500 mL of boiling 0.1% [w/v] SDS and slow cooling to room temperature with mild agitation.

1.6.3 Riboprobe Generation

A 268 bp crhC 5′ UTR riboprobe was generated using a Riboprobe System kit (Promega) as instructed by the manufacturer. Under RNase-free conditions, promoter deletion construct DC6 (Table 3) was linearized with Nhe I (Example 1.3.2), phenol: chloroform extracted and ethanol precipitated (Example 1.3.3), and used as the DNA target for transcription by RNA polymerase. A 107 nucleotide (nt) ssRNA control was also generated by performing a Hae III digestion of the vector pGEM3CS and transcribed with SP6 RNA polymerase for riboprobe generation. The following reagents were added to a 20 μL riboprobe reaction: 1× Transcription Optimized Buffer (Promega), 10 mM DTT, 20 units of Recombinant Rnasin Ribonuclease Inhibitor, 500 μM of each rATP, rCTP, rGTP, 12 μM rUTP, 1 μg of linearized Nhe I DC6 or Hae III pGEM3CS, 50 μCi of [α³²P] rUTP, and 20 units of T7 RNA polymerase for the DC6 reaction or SP6 in the case of the pGEM3CS template. The reaction was incubated at 37° C. for 60 minutes. Prior to electrophoresis, 10 μL of 3×SDS loading buffer was added to the sample. The riboprobes were isolated by electrophoreses on an 8 M urea, 10% denaturing polyacrylamide gel (PAGE) (1.7 mL of 30% [w/v] acrylamide, 8 M urea, 500 μL of 10×TBE, 1.75 mL mQdH₂O, 25 μL of 10% [w/v] APS, 3 μL of TEMED). The gel was electrophoresed at 200 V for 30 minutes, visualized by autoradiography after exposure for 1 minute. The radioactive region was excised from the gel and eluted overnight in RNA elution buffer (500 μL) (0.5 M ammonium acetate, 1 mM EDTA, 0.1% [w/v] SDS). The elution product was extracted with phenol: chloroform (Example 1.3.3) and ethanol precipitated at −80° C. For safe-keeping, one tube remained stored at −80° C. and the other tube was resuspended in 50 μL of RNase-free mQdH₂O and Cerenkov counted (Example 1.3.9.1).

1.6.4 Ribozyme Reaction

To determine if the crhC 5′ UTR is a ribozyme (Winkler et al., 2002), a 20 μL self-cleavage reaction was performed. 100-200 fmoles (5000 cpm) of the 5′ UTR crhC riboprobe (Example 1.6.3) was incubated in 1× Ribozyme Reaction buffer (50 mM Tris-HCl, pH 8.5, 100 mM KCl and 20 mM MgCl₂). Three identical reactions were incubated at 37° C., 30° C., or 20° C. for 17-40 hours with shaking. Reaction products were separated on an 8 M urea, 10% [w/v] denaturing polyacrylamide gel (Example 1.6.3). The gel was dried (Example 1.4.1.1) and the cleavage products visualized by autoradiography at −80° C. for 24-48 hours under an intensifying screen.

1.7 Transcriptional Reporter Fusions

The majority of transcriptional lux reporter fusion constructs (Table 5) were constructed by amplifying the indicated insert regions using PCR (Table 2) (Example 1.3.4), digesting the PCR ends with the appropriate restriction enzyme(s) (Example 1.3.2), gel purifying the insert (Example 1.3.3), and ligating (Example 1.3.5) the insert into a compatible, linearized vector containing the lux operon (Table 4). Due to the lack of restriction sites present within the multiple cloning sites (MCS) of pNLP10, pSIG11, and pSIG16, alternate cloning strategies were performed to allow for proper insertion of the desired DNA sequence. pJBm1 and pJBm2 (Table 1.5) were constructed by performing a triple ligation into BamH I cleaved pSIG16(lux⁻) and pSIG11(lux⁻) respectively. A 269 bp upstream region containing the crhC 5′ UTR was amplified using primers JB5:JB6 and cleaved with BamH I/Nhe I. Downstream sequence containing the crhC ORF and 3′UTR was isolated by a BamH I/Nhe I digestion of pWM753R. The BamH I/Nhe I upstream and downstream sequences were ligated together to produce a crhC insert lacking the crhC promoter. Alternate cloning strategies were also employed to construct pJBm5 and pJBm6 (Table 5). The crhC gene lacking the 3′UTR was isolated by a Hinc II/SnaB I digestion of pWM753, producing a 2170 bp insert. Due to incompatible restriction sites present within the MCS of pNLP10, several of the crhC inserts were first cloned into pBluescript (pBS) KS+, cleaved out of KS+ with BamH I/Xho I, and then cloned into BamH I/Xho I cleaved pNLP10.

For promoter studies, the crhC promoter regions were cloned upstream of the lux operon in pNLP10 whereas for mRNA stability studies the crhC 5′ UTR regions were cloned between a constitutive E. coli promoter (pSIG11) and the lux operon (Table 4 and Table 5). Positive clones were identified by restriction digestion (Example 1.3.2) and sequencing (Example 1.3.8), and stored as 15% [v/v] glycerol stocks at −80° C.

1.7.1 Luciferase Assays

Overnight cultures were diluted 1:25 into LB_{Kan} (50 μg/mL) and incubated at 37° C. until an OD₆₀₀ of 0.4-0.6. In replicates of five, a 250 μL aliquot of the culture was transferred to one well of a 96-well clear bottom assay plate (Corning Incorporated 3610) in a 37° C. incubator. The luciferase activity (cpm) and the number of cells (OD₆₀₀) of the optimally grown transcriptional fusion constructs were immediately determined using a Wallac Victor 2 1420 Multilabel Counter (Perkin Elmer Life Science). The remaining culture was transferred to a 20° C. water bath shaker where it was cold-shocked for the indicated times. At the specified times, 250 μL aliquots were removed (in quintuplicate) and the luciferase activity and OD₆₀₀ were determined as mentioned above. An LB medium control was also treated identically to the test samples and used to determine background emissions from the medium alone. For comparative analysis, the corrected luciferase activity (cpm/OD₆₀₀) was determined, taking into account the LB medium background: Corrected luciferase activity=(cpm(construct)−cpm(LB))/(OD₆₀₀(construct)−OD₆₀₀(LB)). The results were plotted graphically as histograms using Microsoft Excel.

The inability of the crhC promoter motifs or the full-length crhC promoter to convey temperature-dependent expression to lux suggested that crhC expression is regulated at multiple levels. Previous work demonstrated that the transcript half-life of crhC increased significantly during growth of Anabaena at reduced temperature (20° C.) suggesting, that the crhC transcript is stabilized in the cold (Chamot and Owttrim, 2000). In support, mRNA stability has also been shown to be a key regulator in the temperature-dependent expression of CspA, the major cold shock protein in E. coli (Yamanaka et al., 1999; Goldenberg et al., 1996). Sequence analysis of CspA and most other cold shock genes identified unusually long (between 100-250 bp) 5′ untranslated regions (UTR), believed to convey mRNA stability during cold stress. Examination of crhC identified a long, 115 bp 5′ UTR which prompted further investigation into the role of mRNA stability in the temperature-dependent differential expression of crhC (Chamot and Owttrim, 2000; Chamot et al., 1999).

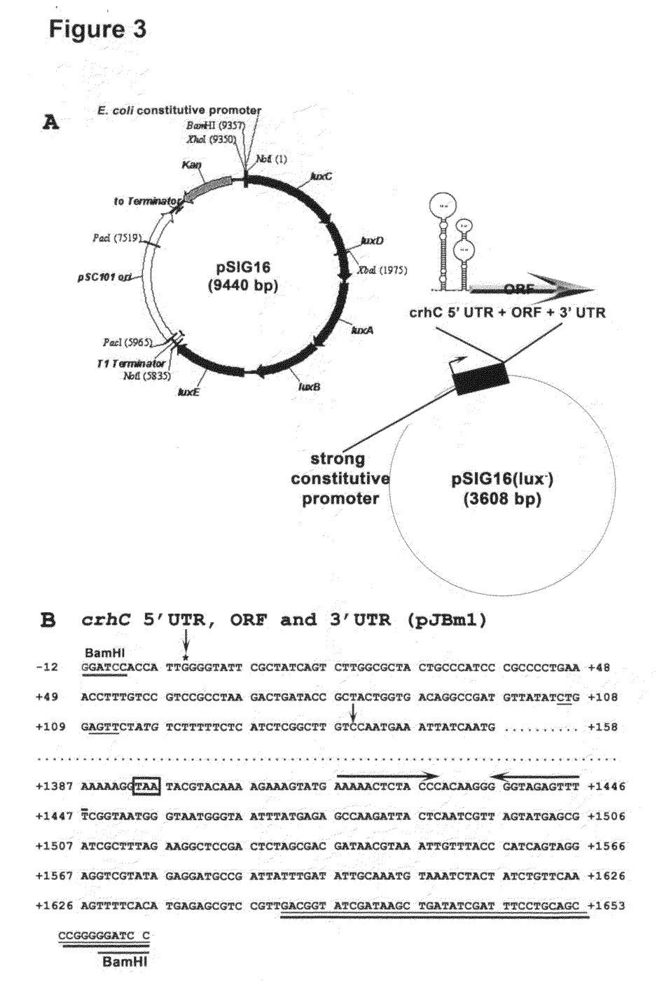

The involvement of post-transcriptional regulation via the 5′ UTR providing temperature-regulated crhC expression was first examined by construction of the plasmids pJBm1 and pJBm2 (Table 5) from the pSIG16 and pSIG11 vector backbones, respectively (FIG. 3A). By removing the lux operon to create pSIG16(lux⁻) and pSIG11(lux⁻) (Table 5), the vectors' strong and medium strength E. coli constitutive promoters were exploited to provide constitutive transcriptional regulation to the crhC gene, in the absence of its own promoter. Using PCR amplification (JB5:JB6) (Table 2) and restriction enzyme digestion, the complete 1664 bp 5′ UTR, ORF and 3′UTR (lacking its own promoter) (FIG. 3B) of crhC was cloned into BamH I cleaved pSIG16(lux⁻) (pJBm1) and pSIG11(lux⁻) (pJBm2), downstream of the respective E. coli constitutive promoters (FIG. 3A). Theoretically, if post-transcriptional regulation via the 5′ UTR was not involved in the temperature-dependent expression of crhC, constitutive expression of crhc, regardless of the growth temperature, would be observed.

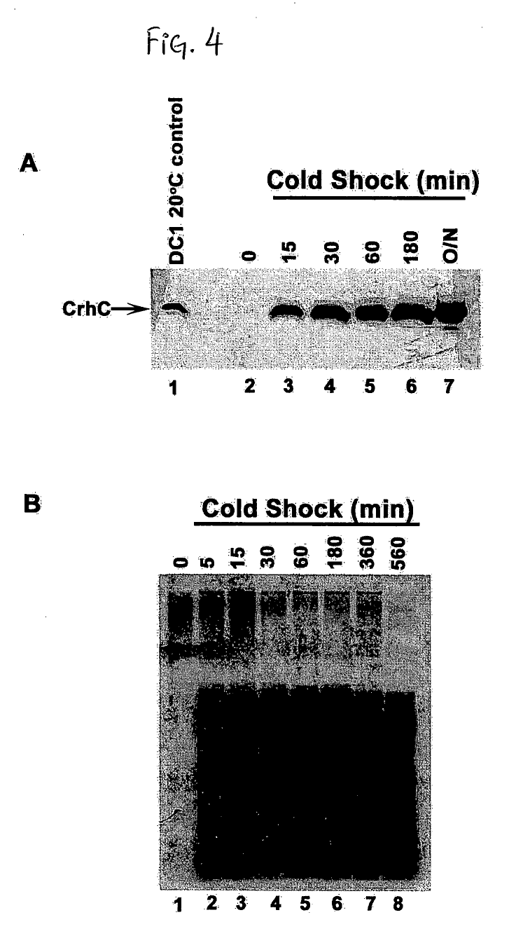

Western and Northern blot analysis of pJBm1 (FIG. 4) and pJBm2 (data not shown) in E. coli demonstrated cold-induced temperature-dependent expression of crhC at both the transcript and protein level. Western blot analysis of pJBm1 E. coli protein lysates probed with anti-CrhC antibody, did not detect CrhC when grown at 37° C. (FIG. 4A, lane 2), compared to the promoter deletion construct DC1 cold stress control (FIG. 4A, lane 1). Upon cold shocking at 20° C., a significant accumulation of CrhC was detected after 15 minutes (FIG. 4A lane 3); with protein levels continuing to increase with lengthened exposure to cold temperatures (FIG. 4A lanes 4-7). A similar cold-induced accumulation pattern was also found for crhC transcript levels. Northern analysis on pJBm1 RNA showed a dramatic increase in crhC transcript accumulation upon a temperature downshift from 37° C. to 20° C. (FIG. 4B lanes 1 and 2). The crhC transcript accumulated after only 5 minutes of exposure to cold stress temperatures (FIG. 4B lane 2), with the transcript levels remaining relatively constant as the exposure time increased (FIG. 4B lanes 3-8). The presence of a RNA smear, indicative of crhC transcript accumulation, is attributed to transcriptional run-on through the pSIG16(lux⁻) plasmid, producing transcript and degradation products varying in length. In conclusion, these results demonstrated that even under the transcriptional control of a strong E. coli constitutive promoter, temperature-dependent expression of crhC still occurred. Overall, the results suggest the involvement of multiple levels of regulation, which primarily include post-transcriptional regulation perhaps mediated by the crhC 5′ UTR.

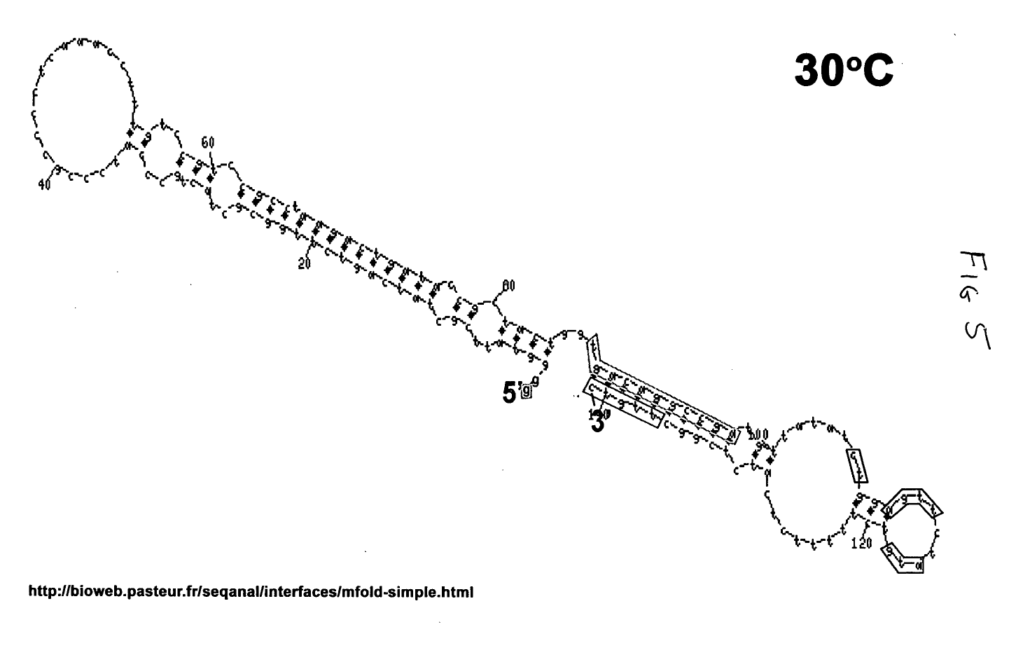

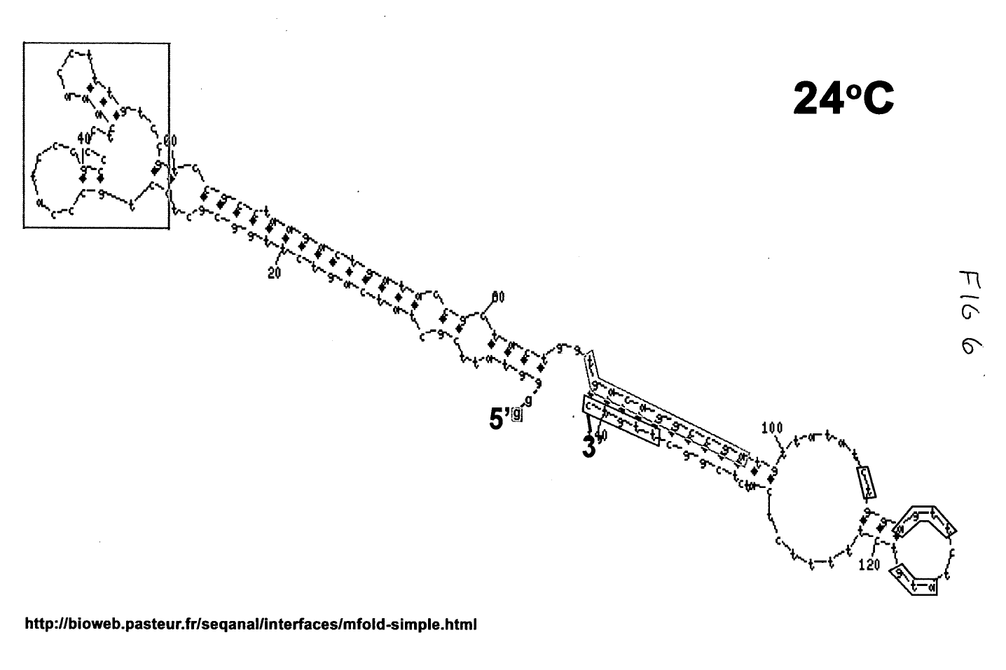

To investigate if RNA secondary structure was providing transcript stability during cold stress, efforts were focused on the crhC 5′ UTR region. The RNA secondary structure of the 5′ UTR was predicted using the computer program MFOLD (http://bioweb.pasteur.fr/seqanal/interfaces/mfold-simple.html). A 141 bp 5′ UTR sequence, starting from the transcriptional start site (+1) and ending 26 bp into the ORF, was used as the query sequence to identify two 5′ UTR stem-loop structures with a AG of −41.9 kcal/mL and −49.1 kcal/mL, predicted to form at 30° C. (optimal) and 20° C. (cold stress) respectively (FIG. 5 and FIG. 6). It should be clearly noted, that it was necessary for the 5′ UTR query sequence to contain at least 26 bp into the crhC ORF for both stem-loop structures to be observed in MFOLD. For example, if the query sequence was shortened to contain only 11 bp into the ORF, as utilized in the promoter-lux transcriptional fusion pJBp4, only the 5′ stem-loop structure was observed and temperature-regulated expression was not observed (data not shown).

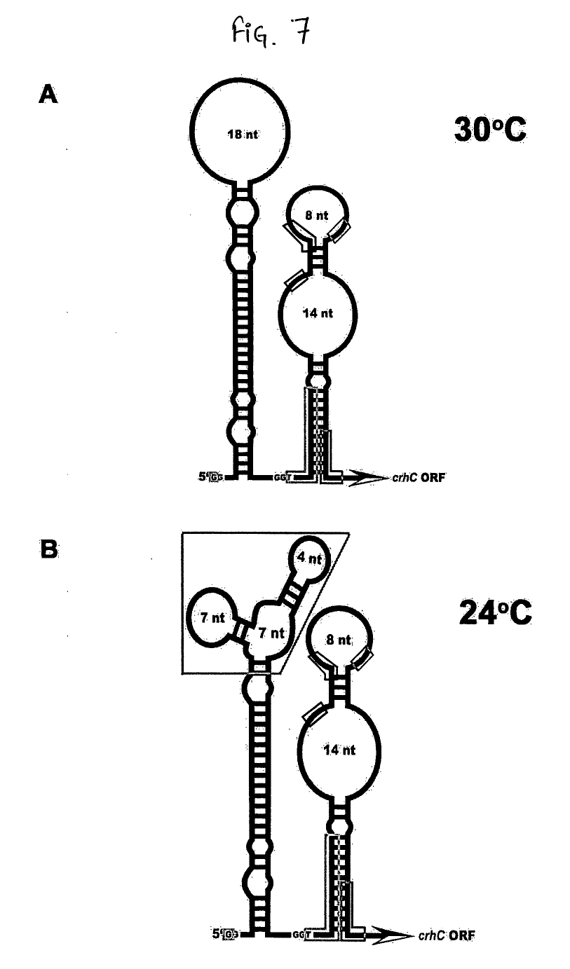

By altering the temperature parameters within MFOLD it was possible to visualize alterations in the 5′ UTR stem-loop structures based solely on thermodynamics. At 30° C., the 5′ stem-loop structure consisted of a 25 bp duplex stem with four small (2-4 nucleotide (nt)) internal loops and an 18 nt hairpin loop (FIG. 5 and FIG. 7A), with a AG of −21.6 kcal/mL. The 3′ stem-loop consisted of a smaller 15 bp duplex stem with two internal loops, 2 nt and 14 nt in size, and an 8 nt hairpin loop, with a ΔG of −18.7 kcal/mol. The 5′ stem has a 56% G-C content and displayed 24 Watson-Crick bonds and 1 wobble base pair (G-U). The 3′ stem-loop contains all of the regulatory elements (indicated by colored boxes), with the stem having a 67% G-C content and displaying 12 Watson-Crick bonds and 3 wobble base pairings (G-U). Upon a temperature downshift, alterations in the 5′ UTR secondary structure arose at temperatures ≦24° C., which is within the Anabaena cold shock range (Chamot et al., 1999). The major changes in secondary structure predicted by MFOLD arose within the loop of the 5′ stem-loop structure (FIG. 6 and FIG. 7B, indicated by the orange box). Between 25° C. and 24° C., the 5′ loop becomes constricted with a AG of −26.4 kcal/mL. The 5′ hairpin loop present at 30° C. (and 25° C.) becomes a 7 nt junction loop with two smaller hairpin loops, 7 nt and 4 nt in size. The 5′ duplex stem also shortens from 25 bp to 22 bp with only 3 small internal loops. No alterations were predicted within the 3′ stem-loop structure where the majority of the regulatory motifs are located however, the ΔG increased to −21.1 kcal/mL. These results suggest that cold-induced alterations within the 5′ UTR secondary structure may provide a regulatory mechanism for differential expression of crhC, by conveying mRNA stability at cold stress temperatures and by destabilizing the transcript at optimal growth temperature.

3.1.1 5′ UTR Luciferase Assays

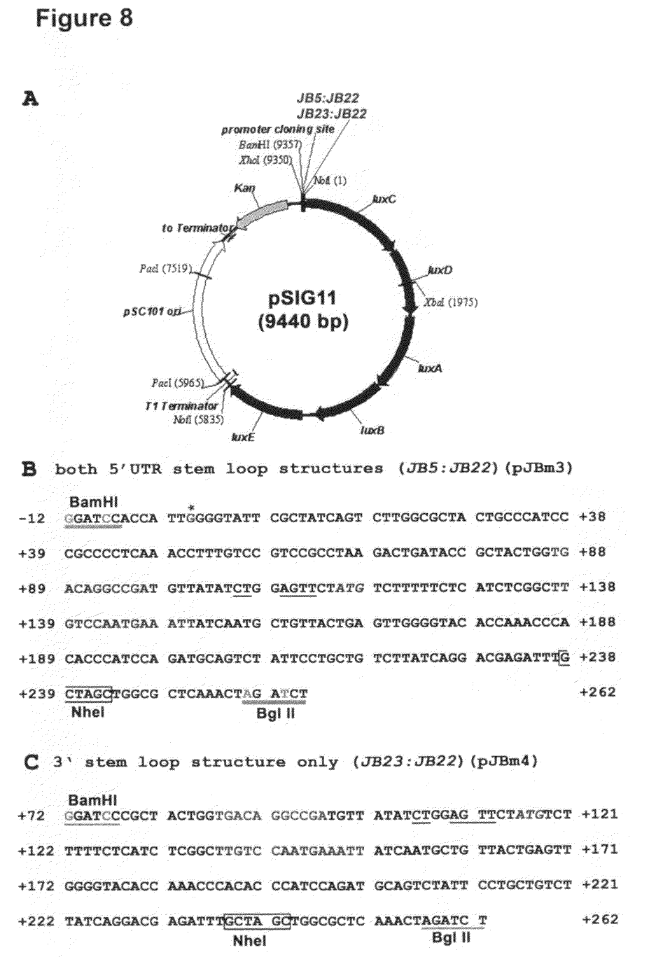

In order to determine if temperature-dependent expression of crhC is regulated post-transcriptionally via mRNA stability conveyed by the 5′ UTR, transcriptional reporter fusions were constructed to determine if the 5′ UTR stem-loop structures could convey temperature-dependent expression to lux. Plasmids pJBm3 and pJBm4 (Table 5) were constructed by cloning both of the 5′ UTR stem-loop structures (JB5:JB22) (FIG. 8B) or only the 3′ stem-loop structure (JB23:JB22) (FIG. 8C) respectively, into pSIG11. The 5′ UTR stem-loop structures were cloned between a medium strength E. coli constitutive promoter and the lux operon (FIG. 8A). The constitutive promoters (PSIG11 and pSIG16) were made using the native E. coli sigma (σ) 70 sequence and random primers to generate a degenerate σ⁷⁰ sequences, which varied in promoter strength from the wildtype (Mike Surette, unpublished). If the 5′ UTR was involved in post-transcriptional regulation, an increase in luciferase activity upon cold stress would be expected as a result of stabilization of the lux transcript by the crhC 5′ UTR.

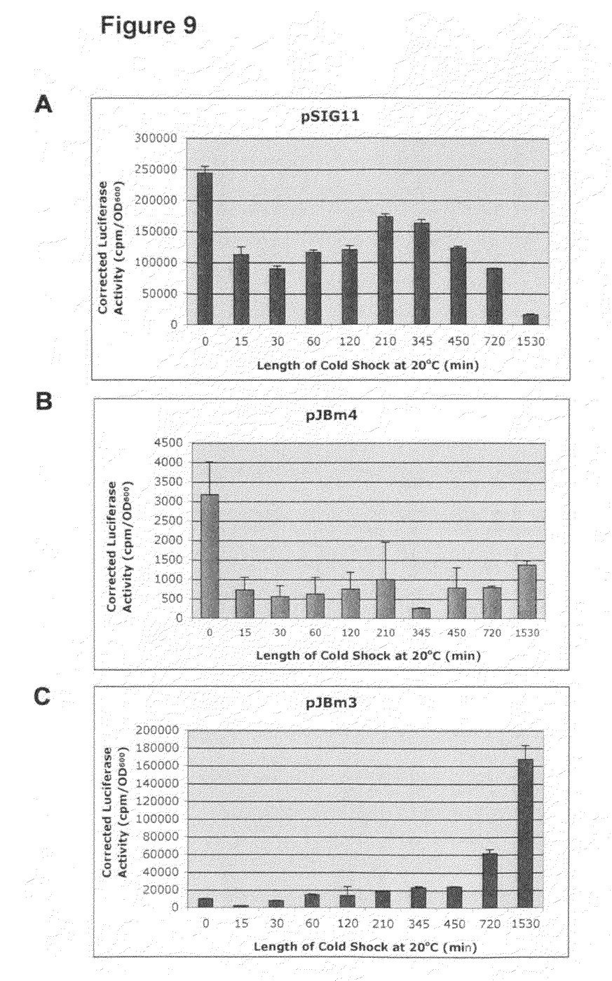

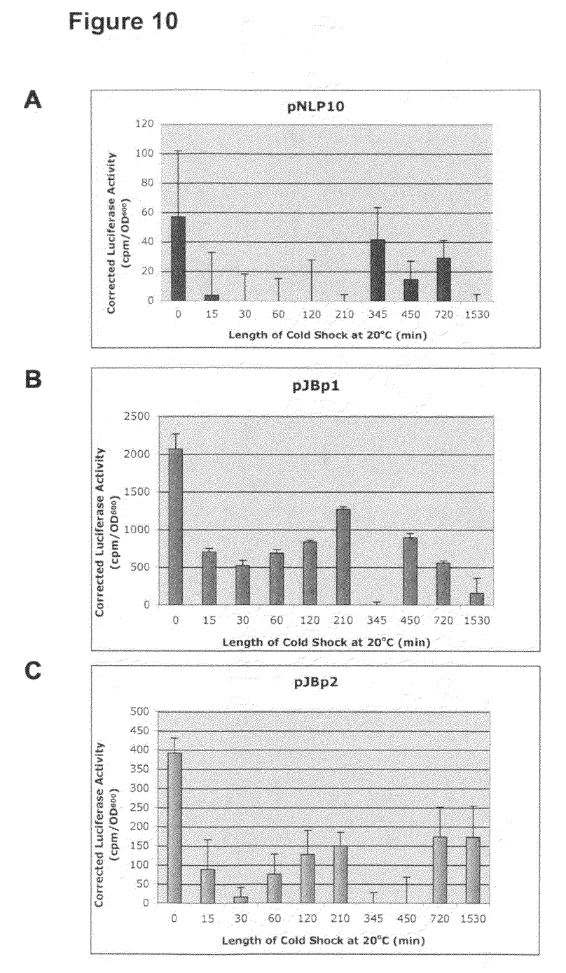

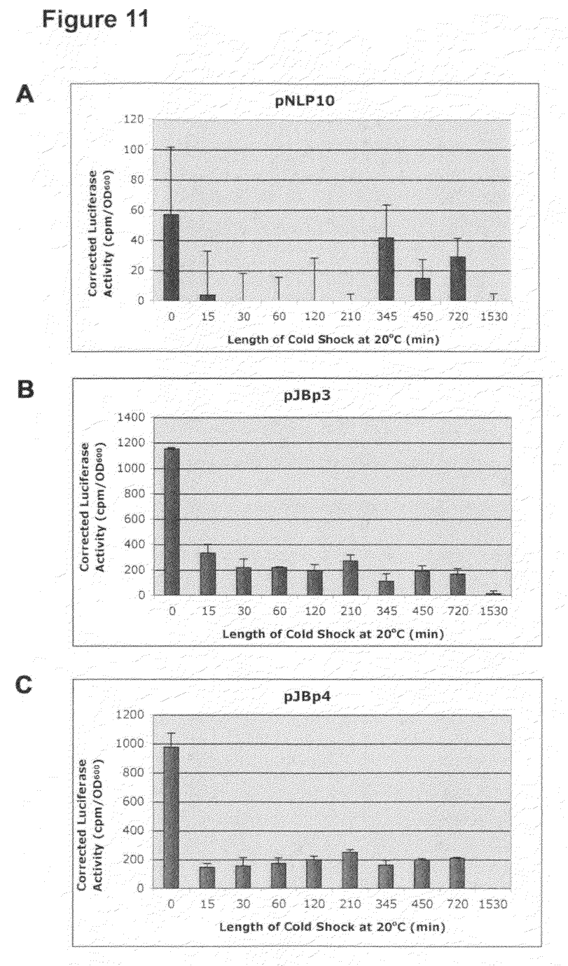

The 5′ UTR-lux transcriptional fusion constructs were grown to exponential phase (37° C.) and subjected to identical cold stress conditions as those performed in the promoter luciferase assays (Example 3.1.11). As illustrated in FIG. 9, a dramatic decrease in luciferase activity was observed for pSIG11, pJBm3, and pJBm4, following transfer to reduced temperature (20° C.). These results are similar to those observed for luciferase activity patterns produced by the promoter transcriptional fusion constructs shown in FIG. 10 and FIG. 11. Although the exact reason(s) for high levels of luciferase activity at 37° C. is not know, these results may indicate that the lux portion of the mRNA may stabilize the transcript at high temperatures, as a similar result was found with a cspA-lacZ fusion (Goldenberg et al., 1996). The drastic decrease in luciferase activity following cold-induction may again be due to an overall drop in cellular enzymatic and metabolic processes (Example 3.1.11). Therefore, to interpret the ability of the 5′ UTR to convey temperature dependence to lux, it is important to analyze the overall pattern of luciferase activity throughout the cold stress time course, in relation to the 37° C. control (0 minutes).

The luciferase activity levels shown in FIG. 9A are indicative of the strength of the E. coli constitutive promoter found in pSIG11. Compared to the crhC promoter (FIG. 11C), the pSIG11 constitutive promoter is 237× stronger, supporting that the crhC promoter activity is relatively weak. Following cold treatment, the pSIG11 luciferase activity levels drop to 51% of that observed at 37° C. (0 minutes), and remained constantly below the 37° C. activity throughout cold stress. As illustrated in FIG. 9B, when only the 3′ stem-loop structure of the crhC 5′ UTR (FIG. 8C) was cloned between the pSIG11 constitutive promoter and the lux operon (pJBm4), no overall change in the luciferase activity pattern was noted throughout the cold stress time course (15 minutes −1530 minutes) however, the luciferase levels were much lower. As expected, upon the initial cold stress induction (15 minutes), pJBp4 luciferase activity decreased 74% (3.8×fold) and remained between 51%-93% (1.1×-14.4×) less than the 37° C. control (0 minutes). These results indicate that the 3′ stem-loop structure of the crhC 5′ UTR is unable to convey temperature-dependent expression to lux.

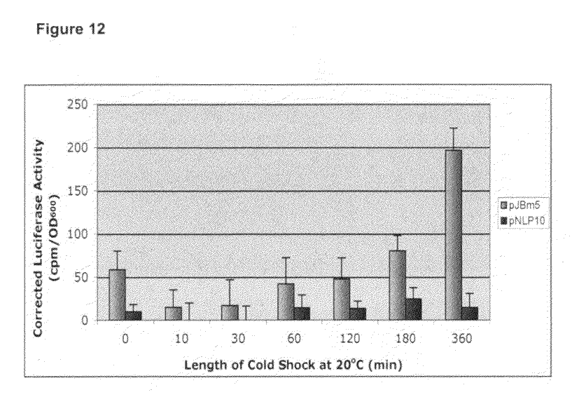

When both stem-loop structures of the crhC 5′ UTR (FIG. 8B) were cloned between the constitutive pSIG11 promoter and the lux operon (pJBm3), a dramatic change in the luciferase activity pattern was noted (FIG. 9C). After 15 minutes of cold stress, a 78% (4.5× fold) decrease in luciferase activity was observed. Importantly, this was followed by an increase in luciferase activity with prolonged exposure to 20° C. The presence of both 5′ UTR stem-loop structures produced a luciferase activity pattern that exceeded the 37° C. control by 1.5×, after 60 minutes of cold stress. Luciferase activity continued to increase up to 18× greater than the 37° C. control, after 1530 minutes at 20° C. In comparison, when the full-length crhC gene was transcriptionally fused to the lux operon only a 1.8× fold increase in luciferase activity was observed after an overnight exposure to 20° C. (FIG. 12). The cold-induced increase in pJBm3 luciferase activity suggests that the crhC 5′ UTR does convey temperature-dependent expression to lux and that both stem-loop structures are required for lux transcript stability at 20° C. In addition, the contribution of mRNA stability conveyed by the 5′ UTR was significantly more important than transcriptional regulation by the crhC promoter, for temperature-regulated expression.

When the actual levels of luciferase activity were monitored (rather then the pattern) between the 5′ UTR transcriptional fusion constructs and the pSIG11 vector control, vast changes in activity were observed. When comparing luciferase levels between pJBm3 and pJBm4, a 1.5× fold increase in luciferase activity was observed when both 5′ UTR stem-loop structures were present (FIGS. 9B and C). This increase in luciferase activity suggests that the presence of both crhC 5′ UTR stem-loop structures stabilizes the lux transcript, thereby increasing Lux protein levels. Interestingly, when comparing the luciferase activity of both pJBm3 and pJBm4 to the pSIG11 vector control, a decrease was noted when either of the 5′ UTR stem loop structures were inserted. The luciferase activity level of the pSIG11 vector 37° C. control (0 minutes) (FIG. 9A) was 25× and 63× greater then when both of the 5′ UTR stem-loop structures or only the second stem-loop structure were inserted, respectively. Although the exact reason is not know, the decrease in luciferase activity at 37° C. when the 5′ UTR sequences are inserted suggests that the presence of the stem-loop structures may destabilize the lux transcript at 37° C. or, the 5′ UTR inserts may decrease the strength of the constitutive E. coli promoter through unknown mechanisms. It is also plausible that the presence of the crhC 5′ UTR may decrease translation thereby limiting the number of Lux products.

The crhC 5′ UTR sequences appear to destabilize the lux transcript in warm conditions, therefore decreasing the overall luciferase levels at 37° C. However, following cold treatment, the lux transcript is stabilized only when both 5′ UTR stem-loops structures are present, permitting luciferase translation and thus activity at 20° C.

3.1.2 Potential Intrinsic Ribozyme Activity of the crhC 5′ UTR

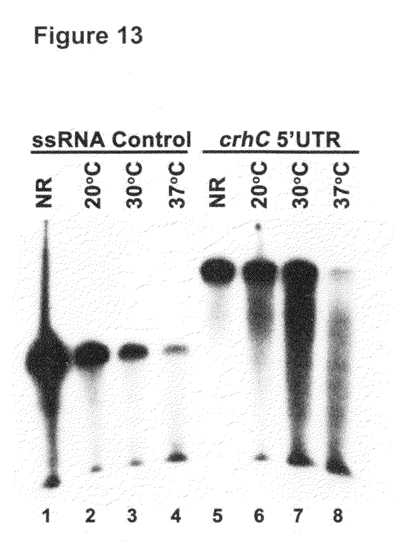

To determine if temperature could induce the crhC 5′ UTR to self-cleave, preliminary ribozyme reactions were performed on the crhC 5′ UTR (268 bp) at various temperatures (Example 1.6.4). Shown in FIG. 13, ribozyme reactions were performed at 37° C., 30° C., and 20° C. (cold stress), with natural self-cleavage being recognized by the presence of a distinct cleavage product(s). A no-reaction (NR) control (FIG. 13 lane 5) was performed without any incubation, to represent the uncleaved, native transcript. A 107 nt ssRNA control transcribed from pGEM3CS, was treated identically to the crhC 5′ UTR, to monitor for contaminating RNase or more likely, spontaneous RNA degradation activity. All RNA is susceptible to spontaneous degradation by alkali attack on the 2′ hydroxyl. As illustrated in FIG. 13 lanes 1 to 4, the control RNA fragments are degraded completely, presumably a result of spontaneous degradation. For all the temperatures tested (FIG. 13 lanes 2-4) a faint smear of small RNA fragments are observed compared to the ssRNA NR control (FIG. 13 lane 1). These results suggest that degradation of the control RNA is temperature independent. Compared to the ssRNA control, variations in the crhC 5′ UTR cleavage patterns and kinetics were observed. As shown in FIG. 13 lanes 6 to 8, distinct smears were observed at various temperatures, indicative of varying degrees of RNA degradation. The NR 5′ UTR control (FIG. 13 lane 5) contained essentially intact 5′ UTR transcript due to the absence of a smear. Following incubation at 20° C. (FIG. 13 lane 6), little degradation of the 5′ UTR was observed, indicated by the intensity of the intact transcript in the upper portion of the gel. These results suggest that at 20° C., the crhC 5′ UTR secondary structure is significantly more stable and not specifically or randomly degraded as compared to 30° C. (FIG. 13 lane 7) or the ssRNA control. Incubation of the 5′ UTR at 30° C. (FIG. 13 lane 7), showed cleavage products arrayed in size however, hints of specific degradation are present within the dark smear. These results suggest that partial degradation or specific cleavage of the 5′ UTR may have occurred at 30° C., producing intermediate sized RNA fragments. Subsequent random cleavage by contaminating RNases would then produce the observed smear. Finally, the 5′ UTR transcripts incubated at 37° C. (FIG. 13 lane 8) had the highest rate of degradation indicated by the presence of a smear. These preliminary results suggest that the crhC 5′ UTR may have self-cleaving properties. The presence of intact 5′ UTR at 20° C. compared to the non-random degradation observed at 30° C. potentially indicates that the crhC 5′ UTR may be a ribozyme, initiating temperature-dependent self-cleavage of the transcript at 30° C. Variations in the degradation rate observed at 30° C. and 37° C. is most likely due to the difference in contaminating RNase activity at the two temperatures. In conclusion, although distinct cleavage products were not observed, preliminary results suggest that the crhC 5′ UTR may possess intrinsic ribozyme activity.

### REFERENCES

The following references are incorporated herein as if reproduced in their entirety.
Ausubel, F. M.; Brent, R.; Kingston, R. E.; Moore, D. D.; Seidman, J. G.; Smith, J. A. and Struhl, K., 1995, Current Protocols in Molecular Biology, John Wiley & Sons, Inc., U.S.A.
Cairrao, F.; Cruz, A.; Mori, H. and Arraiano, C. M., 2003, Mol. Microbiol., 50: 1349-1360.
Chamot, D. and Owttrim, G. W., 2000, J. Bacteriol., 182: 1251-1256.
Chamot, D.; Magee, W.; Yu, E.; and Owttrim, G. W., 1999, J. Bacteriol., 181: 1728-1738.
Feinberg, A. P. and Vogelstein, B., 1983, Anal. Biochem., 132: 6-13.
Goldenberg, D.; Azar, I. and Oppenheim, A. B., 1996, Mol. Microbiol., 19, 241-248.
http://bioweb.pasteur.fr/seqanal/interfaces/mfold-simple.html
Mike Surette, unpublished
Sato, N., 1995, Nucleic Acid Res., 23, 2161-7.
Winkler, W. C.; Nahvi, A. and Breaker, R. R., 2002, Nature, 419: 952-956.
Yamanaka, K.; Mitta, M. and Inouye, M., 1999, J. Bacteriol., 181: 6284-6291.
Yu, E. and Owttrim, G. W., 2000, Nucleic Acid Res., 28: 3926-3934.
Yu, E., 1999, M. Sc. Thesis. University of Alberta, Edmonton, Canada.
Zhen, L. and Swank, R. T., 1993, Biofeedback., 14: 894-898.
Zuker, M.; Mathews, D. H. and Turner, D. H., Algorithms and Thermodynamics for RNA Secondary Structure Prediction: A Practical Guide in RNA Biochemistry and Biotechnology, J. Barciszewski and B. F. C. Clark, eds., NATO ASI Series, Kluwer Academic Publishers, (1999).
Zuker, M. and Stiegler, P., 1981, Nucleic Acid Res., 9: 133-148.

What is claimed is:

1. A method of regulating gene expression in a cell by temperature variation, comprising the steps of:
a) administering to the cell a heterologous nucleic acid molecule, comprising a promoter operatively linked to a 5′-UTR construct of crhC and a coding sequence of the gene; and
b) controlling the temperature environment of the cell.

2. The method of claim 1 wherein the cell is a prokaryotic cell.

3. The method of claim 2 wherein the prokaryotic cell is E. coli or a cyanobacterium.

4. The method of claim 3 wherein the promoter is a constitutive E. coli promoter.

5. The method of claim 4 wherein the promoter is SIG16 or SIG11.

6. The method of claim 1 wherein the coding sequence encodes a heterologous polypeptide.

7. A nucleic acid molecule for regulation of heterologous gene expression comprising a promoter operatively linked to a 5′-UTR construct of crhC and a coding sequence of the gene.

8. The nucleic acid of claim 7 wherein the 5′-UTR construct encodes an RNA having a first secondary structure at a first temperature and a second secondary structure at a second temperature; wherein the first secondary structure is associated with suppression of gene expression while the second secondary structure is associated with active expression of the gene, and wherein the second temperature elicits the cold shock response in Anabaena.

9. The nucleic acid of claim 7 comprising SEQ ID NO:1 or SEQ ID NO:2, or fragments or variants thereof.

10. The nucleic acid of claim 7 which encodes an RNA which functions as a riboswitch, having a secondary structure which produces an active RNA enzyme at the first temperature and not at the second temperature.

11. An expression vector comprising a nucleic acid of claim 7.

12. The expression vector of claim 11 wherein said vector is a plasmid.

13. The expression vector of claim 12 wherein said plasmid comprises pSIG11 or pSIG16.

14. A cell comprising an expression vector of claim 11.

15. The cell of claim 14 which is a prokaryote

16. The cell of claim 15 which is E. coli.

17. The cell of claim 15 which is a cyanobacterium.
